(19) United States
(12) Patent Application Publication
(10) Pub. No.: US 2012/0087919 A1
(43) Pub. Date: Apr. 12, 2012

(54) METHOD FOR TREATING DIABETES

(75) Inventors: Stephan SCHNEIDER, Koln (DE); Ralf SCHIRRMACHER, Montreal (CA)

(73) Assignee: THE ROYAL INSTITUTION FOR THE ADVANCEMENT OF LEARNING/McGILL UNIVERSITY, Montreal (CA)

(21) Appl. No.: 13/223,626

(22) Filed: Sep. 1, 2011

Related U.S. Application Data
(60) Provisional application No. 61/392,727, filed on Oct. 13, 2010.

(30) Foreign Application Priority Data
Feb. 25, 2010 (CA) PCT/CA10/00278

(51) Int. Cl. A61K 39/395; A61P 35/00; A61P 3/00; A61P 3/10; C07K 16/18; C07H 21/04

(52) U.S. Cl. 424/134.1; 530/387.3; 536/23.4

(57) ABSTRACT

Fusion proteins binding specifically to cell types of the islets of Langerhans and delivering therapeutic or prophylactic agents are provided herein, as well as compositions thereof. Methods for treating or preventing diseases related to the pancreas such as diabetes are also disclosed. The therapeutic or prophylactic agents include Nemo-binding domain peptides and other inhibitors of NF-kB activation.

### CROSS-REFERENCE TO RELATED APPLICATIONS

This application is related to International Application No. PCT/CA2010/000278 (published as WO 2010/096930), filed Feb. 25, 2010, which claims priority to U.S. Provisional Application No. 61/155,641 filed Feb. 26, 2009. This application claims priority to U.S. Provisional Application No. 61/392,727 filed Oct. 13, 2010. The contents of these applications are hereby incorporated by reference in their entirety.

### FIELD OF THE INVENTION

This invention relates to fusion proteins comprising single chain antibodies specific for pancreatic β-cells linked to the NEMO-Binding Domain (NBD) peptide, and compositions and methods thereof for the treatment and prevention of diabetes.

### BACKGROUND

Diabetes mellitus affects over 100 million individuals worldwide. In the U.S., the estimated healthcare costs of those affected by diabetes is approximately 136 billion dollars annually. Diabetes mellitus is a disorder of the metabolism that is characterized by the inability of the pancreas to secrete sufficient amounts of insulin, which results in large fluctuations in blood glucose levels and can have both short- and long-term physiological consequences. Long-term complications arising from elevated blood glucose levels (hyperglycemia) in patients with Type I diabetes (insulin-dependent diabetes mellitus, or IDDM) include retinopathy, neuropathy, nephropathy and other vascular complications. Low glucose levels (hypoglycemia) can lead to diabetic coma, seizures, accidents, anoxia, brain damage, decreased cognitive function, and death.

Type II diabetes, also known as non-insulin dependent diabetes mellitus or NIDDM, is a progressive disease characterized by impaired glucose metabolism resulting in elevated blood glucose levels. Patients with type II diabetes exhibit impaired pancreatic beta-cell function resulting in failure of the pancreatic beta-cells to secrete an appropriate amount of insulin in response to a hyperglycemic signal, and resistance to the action of insulin at its target tissues (insulin resistance).

Current treatments of type II diabetes aim to reverse insulin resistance, control intestinal glucose absorption, normalise hepatic glucose production, and improve beta-cell glucose sensing and insulin secretion. Because of the shortcomings of current treatments for diabetes, new treatments for type I and type II diabetes, as well as new diagnostic and prognostic methods, are highly desirable.

Increasing evidence indicates that inadequate β-cell mass underlies type 1 and type 2 diabetes and that apoptosis or programmed cell death is the main form of β-cell death (Weir et al., Diabetes 39:401-405, 1990; Butler et al., Diabetes 52:102-110, 2003). Therefore, strategies for preserving β-cell mass in humans are critically needed. Several studies suggest that β-cell specific activation of the transcription factor NF-κB is a key event in this process (Rehman K K et al., J Biol Chem 278(11):9862-8, 2003; Eldor R et al., Proc Natl Acad Sci USA.103(13):5072-7, 2006). The cytokine-induced activation of the transcription factor NF-κB is an important cellular signal in initiating the cascade of events culminating in β-cell death.

In resting cells, most of the NF-κB-complex is bound to two kinases (IκBα and IκBβ) and the regulatory protein NEMO (NF-κB essential modifier) in the cytoplasm, preventing its translocation to the nucleus and DNA association. Signals from various stimuli (e.g. cytokines, NO) induce phosphorylation, ubiquitination and rapid degradation of IKBs and thereby free NF-κB, which in turn enters the nucleus, binds to DNA and activates transcription. In these cells, intracellular expression of an IκB mutant that is nonphosphorylatable and thus unable to be degraded prevented the nuclear translocation of the NF-κB proteins, even in the presence of cytokines (Jobin, C. et al., J Immunol, 160(1):410-8, 1998).

Moreover, the NEMO-IKBS interaction is required for activation of NF-κB. Blockade by NEMO-binding domain (NBD) peptide leads to inhibition of this process (Mercurio et al., Mol Cell Biol 19(2): 1526-38, 1999). This finding is supported by the report that intraperitoneal injection of NBD ameliorates inflammatory responses in two mouse models of acute inflammation (May, M J et al., Science 289 (5484):1550-4, 2000). Recently, it was shown that the in situ delivery of a transduction-fusion peptide (PTD-5-NBD), by infusion through the bile duct prior to islet isolation, resulted in improved islet function and viability in vitro (Rehman K K et al., J Biol Chem 278(11):9862-8, 2003). However, caveats of this method include the lack of β-cell selectivity as well as the invasive character. Thus, the practical application of this approach to islet transplantation studies and other clinical fields is uncertain.

In recent years, several strategies have been evaluated to deliver NF-κB inhibitors to islet cells in culture or in rodents in situ (e.g. adenoviral-mediated transfer, in situ transduction) with promising results on islet viability and function (Rehman K K et al., J Biol Chem 278(11):9862-8, 2003; Giannoukakis N et al., J. Biol. Chem. 275(47):36509-13, 2000). However, as yet none of these approaches has allowed for non-invasive and β-cell specific delivery in vivo.

Thus, β-cell specific inhibition of NF-κB could be a powerful strategy to prevent β-cell death in the early stages of diabetes development or after islet transplantation. There is a need for novel transport agents exhibiting high specificity for β-cells, in order to permit the specific and non-invasive delivery of a selected therapeutic agent such as an NF-κB inhibitor to islet β-cells within the pancreas.

### SUMMARY OF THE INVENTION

Previously we isolated single-chain antibodies (SCAs) highly specific for pancreatic β-cells in vivo (termed SCA B1-B5; Ueberberg, S. et al., Diabetologia. 53(7):1384-94, 2010; Ueberberg, S. et al., Diabetes 58(10):2324-34, 2009; WO/2010/096930). These SCAs have features indicating that they will be useful for highly selective in vivo delivery to β-cells: rapid (˜5 minutes), specific and high-volume cellular uptake into the cytoplasm of pancreatic β-cells (˜650,000 antibodies/β-cell). Other important characteristics of these SCAs in favor of a therapeutic application are the avid elimination of unbound particles from the circulation (˜20 minutes) and the absence of detectable toxicity to pancreatic β-cells or other cell types. In addition, the biodistribution of one of these SCAs (SCA B1) has been shown to correlate strongly with β-cell mass. These data support the suitability of these SCAs for transport of therapeutic compounds specifically to β-cells in vivo. SCAs highly specific for pancreatic α-cells were also isolated.

We report herein for the first time a non-invasive strategy for islet-cell specific delivery in vivo, and the use thereof to deliver therapeutic agents to islet cells for the prevention and/or treatment of pancreatic diseases such as diabetes. β-cell and/or α-cell specific delivery of therapeutic agents in vivo is provided herein for the first time, using single chain antibodies (SCAs) which specifically bind to cell types of the islets of Langerhans in conjunction with a therapeutic agent, such as a peptide covalently linked to the SCA.

Accordingly, in one embodiment the present invention provides fusion proteins containing single chain antibodies (SCAs) which specifically bind to cell types of the islets of Langerhans linked to the Nemo-binding domain. The fusion proteins of the invention bind specifically to pancreatic α- or β-cells. Analogs, homologs, fragments and variants of the fusion proteins which retain the binding specificity of the original fusion protein are also provided herein, as well as pharmaceutical compositions thereof. Diagnostic, prognostic, theranostic and therapeutic methods using the fusion proteins of the invention for diseases related to the pancreas such as type I and type II diabetes, are also provided.

In one embodiment, there is provided herein a SCA-Nemo-binding domain (NBD) fusion protein, e.g. a SCA B1-NBD fusion protein. We show that the SCA B1-NBD fusion protein can protect islets against the detrimental effects of IL-1β in vitro and against diabetogenic agents in vivo, and enables nearly complete protection from diabetes development in multiple low dose streptozotocin (MLDS)-injected mice. Pharmaceutical compositions comprising the fusion protein and methods of use thereof for the prevention and/or treatment for diseases of the pancreas, such as type I and type II diabetes, are also provided.

Thus, in one aspect, the present invention relates to a fusion protein comprising a single chain antibody (SCA) specifically binding to a cell type of the islets of Langerhans and an agent for the prevention or treatment of a pancreatic condition, or an analog, homolog, fragment or variant thereof which retains the binding specificity of the fusion protein. In an aspect, the SCA and the agent are covalently linked, and may be joined by a linker sequence.

In an embodiment, the agent inhibits NF-κB activation. For example, the agent may be a NEMO-Binding Domain (NBD) peptide, such as the amino acid sequence of SEQ ID NO: 16.

In an embodiment, the sequence of the SCA includes a heavy chain CDR1 amino acid sequence, CDR2 amino acid sequence, and CDR3 amino acid sequence, respectively, of amino acid residues 34-38, 53-69, 102-108 of SEQ ID NO:2; and a light chain CDR1 amino acid sequence, CDR2 amino acid sequence, and CDR3 amino acid sequence, respectively of amino acid residues 159-169, 185-191, 224-232 of SEQ ID NO:2. The SCA may also comprise heavy and light chain CDR1, CDR2 and CDR3 amino acid sequences 34-38, 53-69, 102-108, 159-169, 185-191, and 224-232, respectively, of SEQ ID NO:4, SEQ ID NO:6, SEQ ID NO:8, or SEQ ID NO:10, or an analog, homolog, fragment or variant thereof. In an embodiment, the SCA specifically binds to a β-cell. In another embodiment, the SCA is humanized.

In another embodiment, the present invention provides a fusion protein that specifically binds to a β-cell and selectively inhibits NF-κB activation in the β-cell in vivo.

There are also provided analogs, homologs, fragments or variants of the fusion proteins provided herein, wherein the analogs, homologs, fragments or variants have at least 60%, at least 70%, at least 75%, at least 80%, at least 85%, at least 90%, at least 95% or at least 99% sequence identity to the fusion proteins provided herein. In an aspect, the analogs, homologs, fragments or variants retain the binding specificity of the fusion proteins or SCAB described herein.

In another embodiment, the present invention provides a fusion protein comprising the amino acid sequence set forth in SEQ ID NO: 15 or SEQ ID NO: 18, or an analog, homolog, fragment or variant thereof which retains the binding specificity of the fusion protein or of the sequences set forth in SEQ ID NO: 15 or SEQ ID NO:18. A fusion protein consisting of the amino acid sequence of SEQ ID NO: 15 or SEG ID NO: 18 is further provided herein.

In a further embodiment, the fusion proteins of the invention further comprise a linker sequence between the SCA and the agent. For example, the linker sequence may be flexible glycine-serine sequence or may comprise the amino acid sequence set forth in SEQ ID NO: 17.

Nucleic acid molecules encoding the fusion proteins or analogs, homologs, fragments or variants thereof described herein are also provided. Nucleic acid molecules may, for example, be linked to an expression control sequence to form an expression vector, wherein said expression vector is propagated in a suitable cell.

In yet another embodiment, the present invention provides pharmaceutical compositions comprising the fusion proteins or analogs, homologs, fragments or variants thereof described herein and a pharmaceutically acceptable carrier or excipient.

There are also provided herein methods for preventing or treating a pancreatic condition or disease comprising administering a fusion protein or analog, homolog, fragment or variant thereof of the invention to a subject in need thereof. In the methods provided herein, the subject may be for example a rodent, canine, pig, primate or human.

In an embodiment, the condition or disease is a metabolic disorder, for example a β-cell associated disorder. The condition or disease may be Type I diabetes, Type II diabetes or a complication of diabetes, or a cancer, such as an endocrine tumor.

In another embodiment, β-cell degeneration is prevented or inhibited in the subject; the functionality of pancreatic cells is improved or restored in the subject; plasma insulin levels are increased in the subject; and/or the number or size of pancreatic cells is increased in the subject. In a particular embodiment, the pancreatic cells are β-cells.

In yet another embodiment, NF-κB activation is inhibited in the subject, e.g. selectively inhibited in pancreatic β-cells in the subject. In one embodiment, IL-1β-mediated induction of NF-κB is selectively inhibited in pancreatic β-cells in the subject.

In one embodiment, the fusion protein or analog, homolog, fragment or variant thereof is humanized. In another embodiment, the fusion protein is administered by injection, orally, intravenously, intraperitoneally, intramuscularly or subcutaneously.

### BRIEF DESCRIPTION OF THE DRAWINGS

Particular embodiments of the present invention will now be explained by way of example and with reference to the accompanying drawings, in which:

FIG. 1 shows the amino acid sequences of the β-cell specific SCA B1 to B5 (SEQ ID NO: 2, 4, 6, 8 and 10) and the α-cell specific SCA A1 and A2 (SEQ ID NO: 12, 14). Amino acids are shown in one letter codes. Positions which are diverse in the repertoire are marked in bold. Boxes indicate the complementarity determining region (CDR) determined according to Kabat et al., 2000. Heavy and light chain regions are underlined (heavy chain, light chain).

FIG. 2 shows immunofluorescent staining for binding of the SCA to human islets in a representative pancreas from a non-diabetic subject. SCA, green; insulin or glucagon, red; DAPI (nucleus), blue. The SCA B1 co-localized selectively with insulin- (a), but not glucagon-staining (b) cells. The SCA A1 co-localized with glucagon-(c), but not insulin-expressing cells (d). All images were acquired at 40× magnification.

FIG. 3 shows the amino acid sequence of the SCA B1-NBD fusion protein. Amino acid sequences of heavy- (H, red) and light- (L, blue) chain are shown. Boxes indicate the complementarity determining regions (CDRs).

FIG. 4 shows immunofluorescence analyses of the biodistribution of the SCA B1-NBD fusion protein after intravenous injection in a non-diabetic CD rat (n=5 rats per group, 30-40 sections and 60-80 islets per rat). Pancreata from rats intravenously injected with PBS were used as control. In the pancreas, the SCA B1-NBD fusion protein co-localized exclusively with the insulin-expressing beta-cells, whereas no binding to glucagon-expressing cells or exocrine cells was detected (staining in green colour for SCAs with an anti-His antibody, staining in red colour for insulin and glucagon and nuclei were stained with DAPI in blue colour). All images were acquired at 40× magnification.

FIG. 5 shows assessment of viability and glucose-induced insulin secretion following in vivo delivery of SCA B1-NBD and exposure to IL-1β in vitro. CD rats were intravenously injected either with SCA B1-NBD, SCA B1 or PBS 2 h prior to islet isolation. After an overnight culture, islets were incubated in the presence or absence of recombinant rat IL-1β for 24 h, respectively. The viability of the islets was determined by staining with calcein AM and propidium iodide and visualized by fluorescence/confocal microscopy (all images were acquired at 20× magnification). Viable cells in the islets are stained green, whereas dead cells appear red. a, Islets of PBS treated rats; b, Islets of PBS rats+IL-1β; c, Islets of SCA B1 treated rats; d, Islets of SCA B1 treated rats+IL-1β; e, Islets of SCA B1-NBD treated rats; f, Islets of SCA B1-NBD treated rats+IL-1β; g, Percentage of viable cell aggregates over the total was determined by scoring green (black bars) versus red fluorescence (grey bars); N=4 independent experiments. Results are expressed as mean±SEM, *p<0.001 vs. islets of PBS and SCA B1 treated rats; # p=0.0002, +p<0.0001 vs. islets of PBS and SCA B1 treated rats+IL-1β; h, Static glucose-stimulated insulin secretion at low (black bars; 100 mg/dl) and high glucose (grey bars; 350 mg/dI) concentrations; N=4 independent experiments. Results are expressed as mean±SEM, *p<0.05 vs. insulin secretion at low glucose concentration of the respective groups; +p<0.01 vs. islets of PBS and SCA B1 treated rats+IL-1β.

FIG. 6 shows NF-κB p65 transcription factor assay following in vivo delivery of SCA B1-NBD and exposure to IL-1β in vitro. CD-rats were intravenously injected either with SCA B1-NBD, SCA B1 or PBS 2 h prior to islet isolation. After an overnight culture, islets were incubated in the presence or absence of recombinant rat IL-1β for 2 h, respectively. Results are shown as percentage of islets of PBS treated rats where NF-κB activation in the lysates of islets of PBS treated rats was taken to be 100%. Results are expressed as mean±SEM of four independent experiments in duplicate. *p<0.01 vs. islets of PBS treated rats.

FIG. 7 shows monitoring for the appearance of diabetes in MLDS diabetes model. CD mice were treated with either SCA B1-NBD or PBS on a daily basis by tail vein injection 2 h prior to STZ-administration. SCA B1-NBD treatment protects mice from MLDS-induced diabetes. Percentage of hyperglycaemic mice in PBS (bold line; n=9) and SCA B1-NBD (dashed line; n=6) treated animals. *p<0.01.

FIG. 8 shows plasma concentration of glucose (a) and insulin (b) 28 days after the last injection of STZ in SCA B1-NBD (grey bars; n=6) or PBS (black bars; n=10) treated CD mice. The respective concentrations were determined in the basal state and 30 min following intraperitoneal glucose injection. Data are presented as mean±SEM. *p<0.05 or ^{α}p<0.001 vs. basal levels; ⁺p<0.05 or ^{#}p<0.01 vs. the respective concentrations in PBS-treated mice.

FIG. 9 shows histological sections of the pancreas 28 days after the last injection of STZ stained for insulin (red) and glucagon (brown); a, in a PBS-treated CD mouse; b, in a SCA B1-NBD-treated CD mouse. Images were acquired at 400× magnification; and in c, Pancreatic β-cell mass 28 days after the last injection of STZ in PBS (black bar: n=9) or SCA B1-NBD (grey bar; n=6) treated CD mice. Data are presented as mean±SEM. *p<0.01 vs. PBS-treated mice.

Other features and advantages of the present invention will become apparent from the following detailed description. It should be understood, however, that the detailed description and the specific examples while indicating particular embodiments of the invention are given by way of illustration only. Various changes and modifications within the spirit and scope of the disclosure will become apparent to those skilled in the art from this detailed description.

### DETAILED DESCRIPTION OF THE INVENTION

Specific and non-invasive delivery of therapeutic agents to pancreatic β-cells (or α-cells) in vivo has been hampered by the lack of suitable β- or α-cell specific transport agents. Recently, we reported the generation of single-chain antibodies (SCAs) highly specific for β- and α-cells in vivo. We report herein the use of single chain antibodies (SCAs) and analogs, homologs, fragments and variants thereof which are highly specific for β- or α-cells of the pancreas, as transport agents to permit the specific and non-invasive delivery of a selected therapeutic agent to islet β- or α-cells within the pancreas. The present invention provides fusion proteins containing SCAs which are highly specific for either β- or α-cells of the pancreas in conjunction with a selected therapeutic agent for the prevention or treatment of pancreatic diseases such as diabetes. Pharmaceutical compositions containing the fusion proteins of the invention and diagnostic, prognostic, theranostic and therapeutic methods of use thereof are also provided.

As used herein, “β-cells” refer to the fully differentiated insulin-producing β-cells of the islets of Langerhans in the pancreas. Pancreatic β-cells are characterized by their secretion of insulin and typically by their cell surface expression of the islet amyloid polypeptide (IAPP). “α-cells” refer to cells of the islets of Langerhans in the pancreas which make and release glucagon.

We report herein the generation of a fusion protein of a particular SCA (SCA B1) with a NEMO-Binding Domain (NBD) peptide. The SCA B1-NBD fusion protein was cloned in the pIRES-EGFP, expressed in bacteria and purified by metal affinity chromatography. Subsequently, the SCA B1-NBD was intravenously injected in rodents and evaluated for its ability to protect islets against diabetogenic agents in vitro and in vivo. As detailed below, we show that the SCA B1-NBD fusion protein binds highly selectively to rat β-cells in vivo. Moreover, SCA B1-mediated in vivo delivery of the NBD peptide prior to islet isolation in rats, improved islet viability after islet isolation and completely blocked IL-1β-mediated induction of NF-κB as well as islet dysfunction in culture. Importantly, repeated intravenous injection of SCA B1-NBD, prior to multiple low-dose streptozotocin (MLDS) administration, induced a striking resistance to diabetes development and preserved β-cell mass in mice. Thus, the fusion protein selectively inhibited NF-κB activation in β-cells in vivo and protected islets against diabetogenic agents.

The development of selective targeting agents for β- and α-cells has been limited due to various hurdles. First, the total β-cell mass constitutes only ˜1-2% of the pancreas, with ˜1 million individual islets being scattered throughout the organ. This mass is even smaller in patients with diabetes. Second, the differences in density and echogenicity between the islets and the exocrine cells of the pancreas are relatively small, thereby limiting the application of conventional ultrasonographic or scintigraphic methods (Meier, J. J. Diabetologia 51, 703-713 (2008)). Third, the general knowledge about potential targets that are exclusively expressed on the α- or β-cell surface is poor.

In order to overcome such limitations, we reported previously the use of phage-display technology to identify single-chain antibodies which bind specifically to β- or α-cells of the pancreas. Phage display is a powerful approach for isolating peptides or antibodies that bind to mammalian cell surface receptors. Repeated phage-panning has been used to isolate human SCAs specific for epitopes important in the medical field for imaging and therapy (Riechmann L. et al. Nature. 332, 323-7 (1988)).

A SCA contains an antigen binding domain in one polypeptide instead of two as is the case for a complete antibody. The antigen binding domain of an antibody comprises two separate regions: a heavy chain variable domain (V_{H}) and a light chain variable domain (V_{L}: which can be either V_{κ} or V_{λ}). The antigen binding site itself is formed by six polypeptide loops: three from V_{H} domain (H1, H2 and H3) and three from V_{L} domain (L1, L2 and L3).

A diverse primary repertoire of V genes that encode the V_{H} and V_{L} domains is produced by the combinatorial rearrangement of gene segments. The V_{H} gene is produced by the recombination of three gene segments, V_{H}, D and J_{H}. In humans, there are approximately 51 functional V_{H} segments, 25 functional D segments and 6 functional J_{H} segments, depending on the haplotype. The V_{H} segment encodes the region of the polypeptide chain which forms the first and second antigen binding loops of the V_{H} domain (H1 and H2), whilst the V_{H}, D and J_{H} segments combine to form the third antigen binding loop of the VH domain (H3). The V_{L} gene is produced by the recombination of only two gene segments, V_{L} and J_{L}. In humans, there are approximately 40 functional V_{κ} segments, 31 functional V_{λ} segments, 5 functional J_{κ} segments and 4 functional J_{λ}segments, depending on the haplotype (Williams S. C. et al., J Mol Biol. 264, 220-32 (1996); Corbett S. J. et al., J Mol Biol. 270, 587-97 (1997)). The V_{L} segment encodes the region of the polypeptide chain which forms the first and second antigen binding loops of the V_{L} domain (L1 and L2), whilst the V_{L} and J_{L} segments combine to form the third antigen binding loop of the V_{L} domain (L3).

Antibodies selected from this primary repertoire are believed to be sufficiently diverse to bind almost all antigens with at least moderate affinity. High affinity antibodies are produced by “affinity maturation” of the rearranged genes, in which point mutations are generated and selected by the immune system on the basis of improved binding.

Analysis of the structures and sequences of antibodies has shown that five of the six antigen binding loops (H1, H2, L1, L2, L3) possess a limited number of main-chain conformations or canonical structures. The main-chain conformations are determined by (i) the length of the antigen binding loop, and (ii) particular residues, or types of residue, at certain key positions in the antigen binding loop and the antibody framework. Analysis of the loop lengths and key residues has allowed the prediction of the main-chain conformations of H1, H2, L1, L2 and L3 encoded by the majority of human antibody sequences (Chothia C. et al., J Mol Biol. 227, 799-817 (1992)). Although the H3 region is much more diverse in terms of sequence, length and structure due to the use of D segments, it also forms a limited number of main-chain conformations for short loop lengths which depend on the length and the presence of particular residues, or types of residue, at key positions in the loop and the antibody framework (Shirai H. et al., FEBS Lett. 399, 1-8 (1996)).

Using phage-display technology, we identified a single chain antibody (SCA) which binds to a β-cell specific epitope and a single chain antibody which binds to an α-cell specific epitope, and showed that these antibodies and their analogs, homologs, fragments and variants can be used for in vivo imaging of β- or α-cell mass (Ueberberg, S. et al., Diabetologia. 53(7):1384-94, 2010; Ueberberg, S. et al., Diabetes 58(10):2324-34, 2009; WO/2010/096930). Such in vivo imaging of islet cell mass has a variety of uses, for example to diagnose diseases of the pancreas such as type I or type II diabetes, to monitor disease progression or to evaluate efficacy of therapeutic regimens.

We now report that the β- or α-cell specific SCAs can be used as transport agents to permit the specific and non-invasive delivery of a selected therapeutic agent to islet β- or α-cells within the pancreas. We report herein the generation of fusion proteins containing β- or α-cell specific SCAs linked to a therapeutic agent, and that the fusion proteins are able to target the β- or α-cells specifically and deliver the therapeutic agent to the cells. In one aspect, therefore, there are provided herein fusion proteins containing SCAs specific for β- or α-cells within the pancreas which are linked to a therapeutic agent for the prevention or treatment of pancreatic diseases.

It should be understood that any therapeutic agent which can be linked or coupled to the SCAs described herein is contemplated for use in the present invention. In one aspect, the therapeutic agent is an agent which preserves β-cell mass, for example by blocking cell death or apoptosis of β-cells, protecting islets against the detrimental effects of IL-1β, protecting against diabetogenic agents, and/or otherwise protecting or improving islet viability and/or function. A therapeutic agent used in the fusion proteins of the invention may also reverse insulin resistance, control intestinal glucose absorption, normalise hepatic glucose production, and/or improve beta-cell glucose sensing and insulin secretion. In one embodiment, the therapeutic agent used in the fusion proteins of the invention may be an inhibitor of the transcription factor NF-κB, or an inhibitor of the cytokine-induced activation of the transcription factor NF-κB. In an embodiment, the therapeutic agent is, or comprises a portion of, the Nemo-binding domain (NBD) peptide. In another embodiment, the therapeutic agent comprises the amino acid sequence set forth in SEQ ID NO: 16.

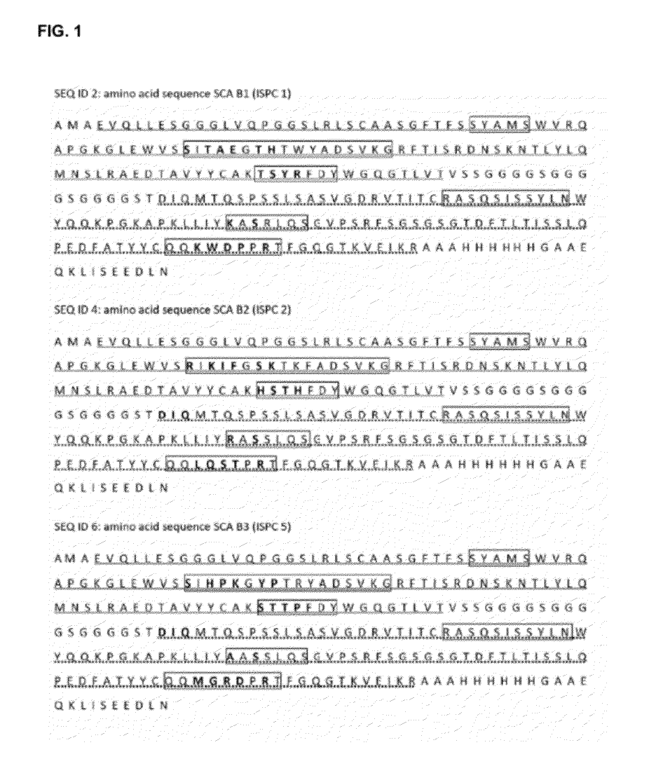

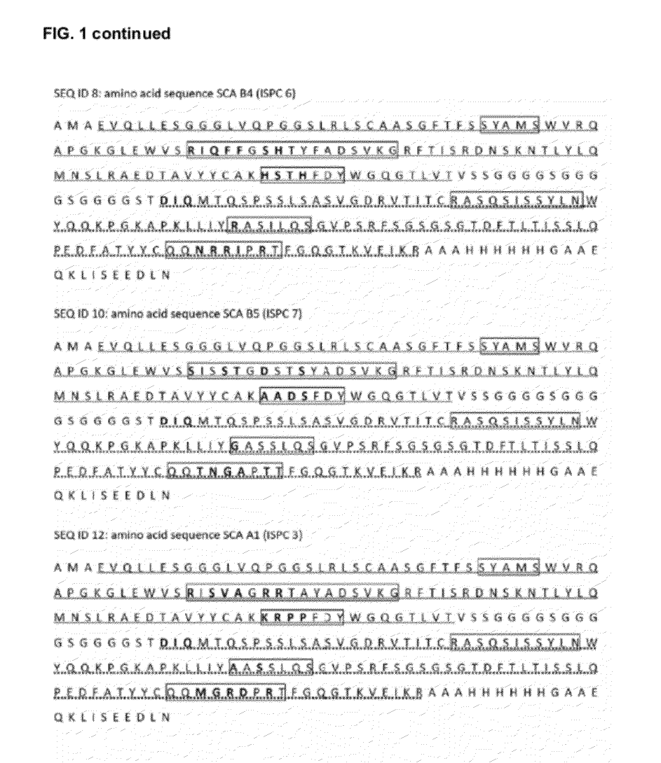

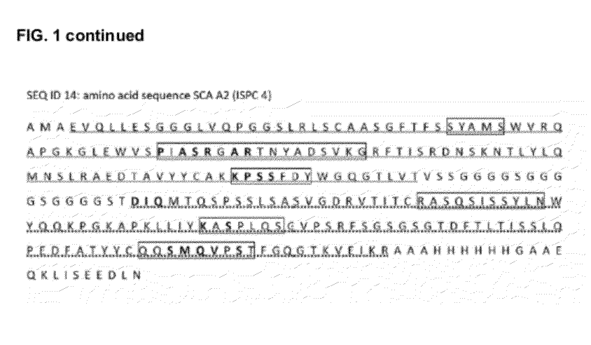

It should also be understood that any SCA which is specific for or α-cells within the pancreas, as well as analogs, homologs, fragments and variants thereof which retain the binding specificity of the original SCA, are contemplated for use in the fusion proteins of the invention. Thus in one aspect, the fusion proteins of the invention comprise an SCA that binds to an epitope on the islets of Langerhans of the pancreas, comprising a heavy chain and a light chain variable region amino acid sequence, each variable region amino acid sequence comprising a contiguous amino acid sequence from within an FR1 sequence through an FR3 sequence that comprises at least one of the amino acid substitutions in the HCDR2 sequences, the HCDR3 sequences, the LCDR2 sequences, and the LCDR3 sequences shown in FIG. 1. In one embodiment, the SCAs bind to an epitope on glucagon secreting α-cells. In another embodiment, the SCAs bind to an epitope on insulin secreting p-cells. In yet another embodiment, the SCAs have complementarity determining regions. In a further embodiment, the β-cell specific SCA has the amino acid sequence shown in SEQ ID NO: 2 or an analog thereof, including but not limited to the analogs shown in SEQ ID NO: 4, 6, 8, and 10 or homologs thereof. In another embodiment, the α-cell specific SCAs have the amino acid sequence shown in SEQ ID NO: 12 or an analog thereof, including but not limited to the analog shown in SEQ ID NO: 14 or a homolog thereof.

The fusion proteins of the invention may optionally comprise a linker sequence which joins the SCA to the therapeutic agent. In one embodiment, the linker sequence is the peptide set forth in SEQ ID NO: 17. It is well-known in the art that many linker sequences may be used in a fusion protein. The choice of linker sequence will be determined by the skilled artisan using techniques which are commonly-known in the art and may depend, for example, on the amino acid sequences of the SCA and therapeutic portions of the fusion protein.

In another aspect, the present invention provides a nucleic acid molecule comprising a nucleic acid sequence encoding the fusion protein of the invention. In one embodiment, the nucleic acid molecule encoding the fusion protein comprises the nucleotide sequence shown in SEQ ID NO:1 or an analog thereof, including but not limited to the analogs shown in SEQ ID NO: 3, 5, 7, and 9 or homologs thereof. In another embodiment, the nucleic acid molecule encoding the fusion protein comprises a nucleotide sequence encoding the peptide set forth in SEQ ID NO: 15 or SEQ ID NO: 16. In another embodiment, the nucleic acid molecule encoding the fusion protein comprises a nucleotide sequence encoding the linker peptide set forth in SEQ ID NO: 17. In a further embodiment, the invention provides an expression vector comprising the nucleic acid operably linked to an expression control sequence. In yet another embodiment, the present invention provides a cell comprising the expression vector or progeny of such a cell, wherein the cell expresses the fusion protein.

In one embodiment, the fusion protein of the invention comprises a β-cell specific SCA, such as SCA B1, B2, B3, B4 or B5. In another embodiment, the fusion protein of the invention comprises an α-cell specific SCA, such as SCA A1 or A2. In a further embodiment, the fusion protein of the invention comprises an analog, homolog, fragment or variant of any of the fusion SCAs described herein which retains the binding specificity of the original SCA.

The proteins and compositions of the invention are useful for treating or preventing conditions where specific and non-invasive targeting of therapeutic or prophylactic agents to cell types of the islets of Langerhans is desired. For example, the proteins and compositions of the invention can be used for treating or preventing conditions or diseases of the pancreas. Non-limiting examples of such conditions or diseases include metabolic disorders, or conditions such as type 1 and type 2 diabetes mellitus, complications of diabetes (such as e.g. retinopathy, nephropathy or neuropathies, diabetic foot, ulcers, macroangiopathies), metabolic acidosis or ketosis, reactive hypoglycaemia, hyperinsulinaemia, glucose metabolic disorder, insulin resistance, metabolic syndrome, dyslipidaemias of different origins, atherosclerosis and related diseases, obesity, high blood pressure, chronic heart failure, edema and hyperuricaemia.

In another aspect, the proteins and compositions of the invention are suitable for preventing β-cell degeneration such as e.g. apoptosis or necrosis of pancreatic β-cells. The proteins and compositions of the invention are also suitable for improving or restoring the functionality of pancreatic cells, and also of increasing the number and size of pancreatic β-cells. In an embodiment, a β-cell associated disorder, including but not necessarily limited to, an endocrine tumor, is treated or prevented by the proteins and compositions of the invention. In a particular embodiment, the condition or disease being treated is a β-cell associated disorder. In one embodiment, the condition or disease is diabetes, particularly type I diabetes, type II diabetes preclinical type I diabetes, and/or diabetic complications.

Thus, in one aspect there is provided herein a method for treating or preventing a metabolic disorder in a subject in need thereof, comprising administering a therapeutically-effective amount of a protein or composition of the invention to the subject. In another aspect, there is provided a method for treating or preventing diabetes in a subject in need thereof, comprising administering a therapeutically-effective amount of a protein or composition of the invention, e.g. the SCAB1-NBD peptide, to the subject.

In yet another aspect, a method for preventing the degeneration of pancreatic β-cells and/or for improving and/or restoring the functionality of pancreatic β-cells in a subject in need thereof, comprising administering a therapeutically-effective amount of a protein or composition of the invention to the subject, is provided. In one aspect, the number or size of pancreatic cells, e.g. β-cells, is increased in the subject, and/or plasma insulin levels are increased in the subject.

In one aspect, the proteins and compositions of the invention inhibit the NF-kB pathway, e.g. inhibit the activation of NF-kB. Accordingly, there is provided herein a method of inhibiting the NF-kB signaling pathway or inhibiting the activation of NF-kB comprising contacting an eukaryotic cell with a protein or composition of the invention. There are also provided methods of modulating or treating a disorder regulated by the NF-kB signaling pathway in a subject in need thereof, comprising administering an effective amount of a protein or composition of the invention to the subject. Methods of regulating cell proliferation or apoptosis in islet cells in a subject in need thereof, comprising administering an effective amount of a protein or composition of the invention, are further provided. In yet another aspect, there is provided a method of selectively inhibiting NF-κB activation in β-cells in vivo in a subject in need thereof, comprising administering proteins and compositions of the invention to the subject.

In a further aspect, there is provided a method of protecting islet cells against diabetogenic agents in vitro and/or in vivo, comprising contacting an eukaryotic cell with, or administering to a subject, a protein or composition of the invention. In an embodiment, islet viability is improved, and/or mediated induction of NF-κB is blocked, and/or islet dysfunction is blocked, and/or β-cell mass is preserved in the subject after administration of a protein or composition of the invention.

In another aspect, the disclosure provides a method for determining the α- or β-cell mass in the pancreas of a subject in need thereof by administering to the subject or to an organ isolated from the subject an effective amount of a fusion protein of the invention in order to diagnose a disease (e.g. type I or type II diabetes), to determine the therapeutic effect of a medicament, or to monitor the transplantation of β-cell mass from a xenogenic or allogeneic source.

In one embodiment, the subject can be a rodent, a canine, a pig, a primate or a human. Although methods of the present invention can be used in any mammal, the subject is preferably a human.

In another embodiment, the therapeutic effect of a medicament or drug can be determined by measuring a decline of β-cell mass in a tumor (including but not limited to insulinoma or nesidioblastosis) or an increase in β-cell mass in the treatment of diabetes type I or type II, using a fusion protein of the invention as an imaging agent. In a further embodiment, the increase of β-cells from xenogeneic or allogeneic sources can be determined as successful β-cell transplantation. In yet another embodiment, the increase of β-cells can be determined during onset of obesity.

In another embodiment, the therapeutic effect of a medicament or drug can be determined as a decline of α-cell mass in a tumor (including but not limited to glucagonoma). In another embodiment a steady α-cell mass can serve as a control for a varying β-cell mass of the same islet of Langerhans.

The present invention also provides methods for diagnosing a metabolic or endocrine disorder in a subject by administering to the subject an effective amount of a fusion protein; obtaining one or more computerized image(s) of at least a portion of the pancreas of the subject; quantitatively analyzing the computerized image(s) in order to determine the α- or β-cell mass in the pancreas of the subject; and comparing the α- or β-cell mass with a baseline measure of α- or β-cell mass, where decreased α- or β-cell mass or increased α- or β-cell mass versus the baseline measure is associated with the presence of a metabolic or endocrine disorder. In another embodiment of the present invention, the computerized image is obtained using PET, SPECT, bioluminescence, magnetic-resonance imaging (MRI), or sonography.

In one embodiment of the invention, SCA B1 or analogs, homologs, fragments or variants thereof are labeled with radioactive isotopes to form a radioligand used to generate the computerized image obtained using PET and SPECT. In another embodiment of the invention, the metabolic disorder is a β-cell associated disorder, including but not necessarily limited to an endocrine tumor. In another embodiment, the β-cell associated disorder is diabetes. In a particular embodiment of the invention, the metabolic disorder is type I diabetes. In another embodiment, the metabolic disorder is type II diabetes. In a further embodiment, the metabolic disorder is preclinical type I diabetes.

In one embodiment of the invention, fusion proteins of the invention or analogs, homologs, fragments or variants thereof are labeled with radioactive isotopes to form a radioligand used to generate the computerized image obtained using PET and SPECT. In another embodiment of the invention, the metabolic disorder is an α-cell associated disorder, including but not necessarily limited to, a glucagonoma.

The present invention additionally provides methods for assessing the prognosis of a subject at risk for developing diabetes by periodically administering to the subject an effective amount of a fusion protein of the invention or its analog or homolog radioligands; obtaining one or more computerized image(s) of at least a portion of the pancreas of the subject; quantitatively analyzing the computerized image(s) in order to determine the β-cell mass in the pancreas of the subject; and comparing the periodically determined β-cell mass with a baseline measure of β-cell mass, where decreased β-cell mass versus the baseline measure is associated with the progression from a prediabetic condition to a diabetic condition. In an embodiment of the invention, the radioligand is SCA B1-NBD peptide or analogs, homologs, fragments or variants thereof and the computerized image is obtained using PET. In another embodiment of the present invention, the subject is at risk for developing type I or type II diabetes.

The present invention also provides methods for determining the efficacy of therapy of a metabolic disorder by periodically administering to the subject an effective amount of a fusion protein of the invention or analogs, homologs, fragments or variants thereof, for example as a radioligand; obtaining one or more computerized image(s) of at least a portion of the pancreas of the subject; quantitatively analyzing the computerized image(s) in order to determine the β-cell mass in the pancreas of the subject; and comparing the periodically determined β-cell mass with a baseline measure of β-cell mass, where a β-cell mass generally equivalent to the baseline measure is indicative of successful therapy to treat the metabolic disorder. In an embodiment of the invention, the radioligand is SCA B1-NBD peptide or analogs, homologs, fragments or variants thereof and the computerized image is obtained using PET. In a further embodiment of the invention, the metabolic disorder is a pancreatic β-cell associated disorder. In a specific embodiment, the β-cell disorder is an endocrine tumor. In yet another specific embodiment, the β-cell disorder is diabetes. In still other embodiments of the invention, the β-cell disorder is type I diabetes, type II diabetes or preclinical type I diabetes.

The invention also provides methods for managing the treatment or prevention of diabetes by periodically administering to the subject an effective amount of a radioligand; obtaining one or more computerized image(s) of at least a portion of the pancreas of the subject; quantitatively analyzing the computerized image(s) in order to determine the β-cell mass in the pancreas of the subject; and comparing the periodically determined β-cell mass with a baseline measure of β-cell mass, where decreased β-cell mass versus the baseline measure is associated with the need for further therapy. In an embodiment of the invention, the radioligand is SCA B1-NBD peptide and its analogs or homologs and the computerized image is obtained using PET. In one embodiment, the diabetes is type II diabetes. In another embodiment, the diabetes is type I diabetes.

The present invention also provides methods for determining the success of an islet cell transplantation, e.g. a β-cell transplantation, by periodically administering to the subject an effective amount of a SCA B1-NBD peptide or analogs, homologs, fragments or variants thereof, for example as a radioligand. Increase of β-cell mass after transplantation indicates success of a β-cell transplantation, whereas decrease in β-cell mass after transplantation indicates lack of success of a β-cell transplantation.

Thus, the present invention provides fusion proteins and analogs, homologs, fragments and variants thereof for the detection of a β-cell specific epitope. Also provided herein are fusion proteins and analogs, homologs, fragments and variants thereof for the detection of an α-cell specific epitope. Such fusion proteins can be used for in vivo imaging of cells of the pancreas, for example to diagnose disease of the pancreas, to monitor disease progression or to assess efficacy of a therapeutic treatment. Accordingly, the present inventions provides fusion proteins and analogs, homologs, fragments or variants thereof which are specific for the identification of α- or β-cell mass in the pancreas.

The term “ISPC” as used herein refers to islet specific phage clones which have been isolated from a phage library consisting of nucleic acid molecules comprising a first DNA sequence encoding the signal peptide of a bacteriophage protein linked at its 3′ end to a second DNA sequence encoding a tag, wherein the second DNA sequence is linked at its 3′ end to a third DNA sequence encoding an immunoglobulin variable region polypeptide. Such ISPCs are known in the art and have been described previously, for example in published US patent application number US/2004/0202995. In one embodiment, the ISPC nucleic acid sequence is human and has the nucleotide sequence shown in SEQ ID NO:1, or an analog or homolog thereof. In another embodiment, the nucleic acid sequence is human and has the nucleotide sequence shown in SEQ ID NO: 3, 5, 7, 9 or 11, or an analog or homolog thereof.

The term “single chain antibody” or “SCA” as used herein refers to a single chain antibody that has been derived from an ISPC. Single chain antibodies are known in the art.

In one embodiment, there is provided herein a fusion protein comprising a single chain antibody (SCA) called SCA B1 which comprises a human CDR and frame region protein sequences and has the amino acid sequence shown in SEQ ID NO:2 (FIG. 1). In another embodiment, fusion proteins comprising single chain antibodies specific for p-cells are provided herein. Such antibodies are for example selected from a group of analogs including but not limited to SCA B1, SCA B2, SCA B3, SCA B4, and SCA B5.

In one embodiment, there is provided herein a fusion protein comprising a single chain antibody (SCA) called SCA B1 which comprises a human CDR and frame region protein sequences and has the amino acid sequence shown in SEQ ID NO:2 (FIG. 1) and a Nemo-binding domain (NBD) peptide, i.e. a SCA B1-NBD fusion protein. In another embodiment, fusion proteins comprising single chain antibodies specific for β-cells and a Nemo-binding domain (NBD) peptide are provided. Such fusion proteins are for example selected from a group of analogs including but not limited to SCA B1-NBD fusion protein, SCA B2-NBD fusion protein, SCA B3-NBD fusion protein, SCA B4-NBD fusion protein, and SCA B5-NBD fusion protein. In other embodiments, fusion proteins comprising single chain antibodies specific for β-cells and the amino acid sequence set forth in SEQ ID NO: 16 are provided. In a particular embodiment, the fusion protein comprises the amino acid sequences shown in SEQ ID NO:2 and SEQ ID NO: 16, or an analog, homolog, fragment or variant thereof. In a further embodiment, the fusion protein further comprises a linker sequence which joins the amino acid sequences shown in SEQ ID NOs: 2 and 16. For example, the fusion protein may further comprise the amino acid sequence set forth in SEQ ID NO: 17.

Thus in an embodiment, the fusion proteins of the invention comprise and SCA specific for pancreatic islet cells covalently linked to an agent for the prevention or treatment of a pancreatic condition, e.g. a NBD peptide, e.g. a peptide having the amino acid sequence set forth in SEQ ID NO: 16. The SCA and the agent may be joined by a suitable linker sequence. It will be appreciated by persons of skill in the art that many linker sequences are possible and can be easily selected using art-recognized techniques. Non-limiting examples of suitable linker sequences include flexible glycine-serine sequences and the amino acid sequence set forth in SEQ ID NO: 17. In an embodiment, the agent, e.g. the NBD peptide, is covalently linked to the C-terminus of the SCA via a linker peptide. In another embodiment, the agent may be covalently linked to the N-terminus of the SCA via a linker peptide.

In an embodiment, there is provided herein a fusion protein having the sequence set forth in SEQ ID NO: 15 or an analog, homolog, fragment or variant thereof. In another embodiment, there is provided herein a fusion protein having the sequence set forth in SEQ ID NO: 18 or an analog, homolog, fragment or variant thereof.

In one embodiment, there is provided herein a fusion protein comprising a single chain antibody SCA A1 which comprises a human CDR and frame region protein sequences and has the amino acid sequence shown in SEQ ID NO: 12 (FIG. 1). In another embodiment, fusion proteins comprising single chain antibodies specific for a α-cells are provided herein. Such SCAs are for example selected from a group of analogs including but not limited to SCA A1 and SCA A2.

The term “homolog” is used to mean those amino acid or nucleic acid sequences which have slight or inconsequential sequence variations from the sequences of the fusion proteins or SCAs described herein, e.g. the sequences set forth in SEQ ID NOs: 2, 4, 6, 8, 10, 12, 14, 15, 16, 17 and 18, i.e., the sequences function in substantially the same manner. The sequence variations may be attributable to local mutations or structural modifications. Sequences having substantial homology include nucleic acid sequences having at least 65%, more preferably at least 85%, and most preferably 90-95% identity with the sequences provided herein, e.g. the sequences shown in SEQ ID NOs: 1, 3, 5, 7, 9, 11, and 13. Sequence identity can be calculated according to methods known in the art. Nucleic acid sequence identity is most preferably assessed by the algorithm of BLAST version 2.1 advanced search. A series of programs is available at http://www.ncbi.nlm.nih.gov/BLAST. The advanced blast search (http://www.ncbi.nlm.nih.gov/blast/blast.cgi?Jform=1) may be set to default parameters. (ie Matrix BLOSUM62; Gap existence cost 11; Per residue gap cost 1; Lambda ratio 0.85 default). Examples of references to BLAST searches are: Altschul, S. F. et al., (1990), J. Mol. Biol, 215:403410; Gish, W. & States, D. J. (1993), Nature Genet. 3:266272; Madden, T. L. et al., (1996), Meth. Enzymol. 266:131-141; Altschul, S. F. et al., (1997), Nucleic Acids Res. 25:33893402; and Zhang, J. & Madden, T. L. (1997), Genome Res. 7:649656.

The term “analog” is used to mean an amino acid or nucleic acid sequence which has been modified as compared to the sequence of the fusion proteins or SCAs described herein, wherein the modification does not alter the utility of the sequence (e.g. the binding specificity of the amino acid sequence or the encoded amino acid sequence, in the case of a nucleic acid sequence) as described herein. The modified sequence or analog may have improved properties over the fusion proteins or SCAs described herein, e.g. sequences shown in SEQ ID NOs:1-18.

Also encompassed are sequences that hybridize to the complement of sequences shown in SEQ ID NO:1 or 13 or a fragment thereof, or that hybridize to the complement of a nucleotide sequence encoding a fusion protein of the invention, and that encode peptides which maintain the property of binding a β- or an α-cell (i.e. maintain the binding properties of the fusion proteins or SCAs described herein). The term “sequence that hybridizes” means a nucleic acid sequence that can hybridize to a sequence of SEQ ID NO:1 or 13 under stringent hybridization conditions. Appropriate “stringent hybridization conditions” which promote DNA hybridization are known to those skilled in the art, and may be found for example in Current Protocols in Molecular Biology, John Wiley & Sons, N.Y. (1989), 6.3.1-6.3.6. The term “stringent hybridization conditions” as used herein means that conditions are selected which promote selective hybridization between two complementary nucleic acid molecules in solution. Hybridization may occur to all or a portion of a nucleic acid sequence molecule. The hybridizing portion is at least 50% the length with respect to one of the polynucleotide sequences encoding a polypeptide. In this regard, the stability of a nucleic acid duplex, or hybrids, is determined by the Tm, which in sodium containing buffers is a function of the sodium ion concentration, G/C content of labeled nucleic acid, length of nucleic acid probe (I), and temperature (Tm=81.5° C.-16.6 (Log10 [Na+])+0.41(%(G+C)−600/l). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a greater than 95% identity, the final wash will be reduced by 5° C. Based on these considerations, in one embodiment stringent hybridization conditions are defined as: hybridization at 5× sodium chloride/sodium citrate (SSC)/5×Denhardt's solution/1.0% SDS at Tm (based on the above equation) −5° C., followed by a wash of 0.2×SSC/0.1% SDS at 60° C.

The fusion protein may be modified to contain amino acid substitutions, insertions and/or deletions that do not alter the binding properties of the fusion protein. Conservative amino acid substitutions involve replacing one or more amino acids of the fusion protein with amino acids of similar charge, size, and/or hydrophobicity characteristics. When only conservative substitutions are made, it is expected that the resulting analog would be functionally equivalent to the unsubstituted protein. Non-conservative substitutions involve replacing one or more amino acids of the fusion protein with one or more amino acids which possess dissimilar charge, size, and/or hydrophobicity characteristics.

The fusion protein may be modified to make it more therapeutically effective or suitable. For example, the fusion protein of the present invention may be converted into a pharmaceutically-acceptable salt by reacting with inorganic acids such as, for example, hydrochloric acid, sulphuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benzenesulphonic acid, and toluenesulphonic acids, for example. Pharmaceutically-acceptable salts are well-known in the art and pharmaceutically-acceptable salts of the fusion proteins and analogs, homologs, fragments and variants thereof are encompassed herein.

The present invention also provides expression vectors comprising a nucleic acid sequence encoding a fusion protein of the invention or a fragment or analog thereof.

Possible expression vectors include, but are not limited to, cosmids, plasmids, artificial chromosomes, viral vectors or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the invention and regulatory sequences selected on the basis of the host cells to be used for expression, operatively linked to the nucleic acid molecule of the invention. “Operatively linked” is intended to mean that the nucleic acid is linked to regulatory sequences in a manner which allows expression of the nucleic acid.

There is provided herein a recombinant expression vector containing a nucleic acid molecule of the invention, or a fragment or analog thereof, and the necessary regulatory sequences for the transcription and translation of the inserted protein-encoding sequence.

Suitable regulatory sequences may be derived from a variety of sources, including bacterial, fungal, viral, mammalian, or insect genes (for example, see the regulatory sequences described in Goeddel, Gene Expression Technology Methods in Enzymology 185, Academic Press, San Diego, Calif. (1990). Selection of appropriate regulatory sequences is dependent on the host cell, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the fusion protein.

The recombinant expression vectors of the invention may also contain a selectable marker gene which facilitates the selection of host cells transformed or transfected with a recombinant molecule of the disclosure.

Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin, such as IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance, transformant cells can be selected with G418. Cells that have incorporated the selectable marker gene will survive, while the other cells die. This makes it possible to visualize and assay for expression of recombinant expression vectors of the disclosure and in particular to determine the effect of a mutation on expression and phenotype. It will be appreciated that selectable markers can be introduced on a separate vector from the nucleic acid of interest.

The recombinant expression vectors provided herein may also contain genes which encode a moiety which provides increased expression of the recombinant protein; increased solubility of the recombinant protein; and/or aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors can be introduced into host cells to produce a transformed host cell. The term “transformed host cell” is intended to include cells that are capable of being transformed or transfected with a recombinant expression vector of the invention. The terms “transduced”, “transformed with”, “transfected with”, “transformation” and “transfection” are intended to encompass introduction of nucleic acid (e.g. a vector or naked RNA or DNA) into a cell by one of many possible techniques known in the art. Prokaryotic cells can be transformed with nucleic acid by, for example, electroporation or calcium-chloride mediated transformation. For example, nucleic acid can be introduced into mammalian cells via conventional techniques such as calcium phosphate or calcium chloride co-precipitation, DEAE-dextran mediated transfection, lipofectin, electroporation, microinjection, RNA transfer, DNA transfer, artificial chromosomes, viral vectors and any emerging gene transfer technologies. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of eukaryotic host cells and prokaryotic cells. For example, the proteins of the disclosure may be expressed in yeast cells or mammalian cells. Other suitable host cells can be found in Goeddel, Gene Expression Technology: Methods in Enzymology 185, Academic Press, San Diego, Calif. (1991). In addition, the proteins of the disclosure may be expressed in prokaryotic cells, such as Escherichia coli (Zhang et al., Science 303(5656): 371-3 (2004)).

Mammalian cells suitable for use in the methods described herein include, among others: COS (e.g., ATCC No. CRL 1650 or 1651), BHK (e.g. ATCC No. CRL 6281), CHO (ATCC No. CCL 61), and HeLa (e.g., ATCC No. CCL 2) and 3T3 mouse fibroblasts (e.g. ATCC No. CCL92).

Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include without limitation pCDM8 (Seed, B., Nature 329:840 (1987)), pMT2PC (Kaufman et al., EMBO J. 6:187-195 (1987)) and pCMV (Clontech, California, U.S.A.).

Alternatively, the fusion proteins of the invention may also be expressed in non-human transgenic animals, such as rats, mice, rabbits, sheep and pigs (Hammer et al. Nature 315:680-683 (1985); Palmiter et al. Science 222:809-814 (1983); Brinster et al. Proc. Natl. Acad. Sci. USA 82:4438-4442 (1985); Palmiter and Brinster Cell 41:343-345 (1985) and U.S. Pat. No. 4,736,866). The present invention also encompasses tissues and cells derived or isolated from such animals.

In addition to the analogs and homologs described above, in certain embodiments, the fusion proteins of the invention may further be recombinantly fused to a heterologous polypeptide at the N- or C-terminus or chemically conjugated (including covalent and non-covalent conjugations) to polypeptides or other compositions. For example, fusion proteins may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387. Any type of molecule may be covalently attached to the fusion proteins of the invention as long as it does not prevent the fusion protein from binding to α-cells or β-cells or alter the binding specificity of the fusion protein (e.g. of the antibody portion of the fusion protein). For example, but not by way of limitation, the fusion protein derivatives include fusion proteins that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids. The heterologous polypeptide to which the antibody is fused may be useful for example to increase the in vivo half life of the fusion protein, or for use in immunoassays using methods known in the art, or for use in diagnostic medical imaging methods as known in the art. Fusion protein of the invention can be fused to marker sequences, such as a peptide to facilitate their purification or detection. In general, it should be understood that fusion protein of the present invention may be used in non-conjugated form or may be conjugated to at least one of a variety of molecules, e.g., to improve the therapeutic properties of the molecule, to improve the pharmacokinetic properties of the molecule, to facilitate target detection, or for imaging in the patient.

In certain embodiments, a fusion protein of the invention includes an additional amino acid sequence or one or more moieties. Exemplary modifications are described in more detail below. For example, the fusion proteins of the invention may comprise a linker sequence, or may be modified to add an additional functional moiety (e.g., PEG, a drug, a toxin, an imaging agent or a label).

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In other embodiments, a polypeptide or amino acid sequence derived from a designated protein may be identical to the starting sequence except for two or fewer, three or fewer, four or fewer, five or fewer, six or fewer, seven or fewer, eight or fewer, nine or fewer, ten or fewer, fifteen or fewer, or twenty or fewer individual amino acid substitutions, insertions, or deletions. In certain embodiments, a polypeptide or amino acid sequence derived from a designated fusion protein has one to five, one to ten, one to fifteen, or one to twenty individual amino acid substitutions, insertions, or deletions relative to the starting sequence or the starting fusion protein.

Also encompassed in the present invention are fragments, derivatives, modifications, or variants of the fusion proteins described herein, as well as the analogs and homologs described above, and any combination thereof. The terms “fragment,” “variant,” “derivative”, “modification”, “homolog” and “analog” when referring to fusion proteins of the present invention include any polypeptides which retain at least some of the antigen-binding properties (e.g. antigen-binding specificity) of the corresponding native fusion proteins. The terms “variant,” “derivative” and “modification” are used interchangeably herein. Fragments of fusion proteins, or of SCAs, of the present invention include proteolytic fragments or deletion fragments. Variants of the fusion proteins of the present invention include fragments, polypeptides with altered amino acid sequences due to amino acid substitutions, deletions, or insertions as described herein, and modifications as described herein. Various derivatives which have been altered so as to exhibit additional features not found on the native polypeptide are also described herein and include, for example, fusion proteins and antibodies conjugated to imaging agents. Variants may occur naturally or be non-naturally occurring (e.g. produced using art-known mutagenesis techniques). Variant polypeptides may comprise conservative or non-conservative amino acid substitutions, deletions or additions as described herein. Derivatives may also have one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

Thus in one embodiment, analogs, homologs, fragments or variants of the fusion proteins disclosed herein are encompassed by the present invention. In an embodiment, the analogs, homologs, fragments or variants retain the antigen-binding specificity and/or properties of the fusion proteins or SCAs disclosed herein.

By “specifically binds,” it is generally meant that an antibody (or other protein, including the fusion proteins of the invention) binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

Alternatively, in another embodiment, mutations may be introduced randomly along all or part of the fusion protein, such as by saturation mutagenesis, and the resultant mutants can be used in the diagnostic and treatment methods disclosed herein and screened for their ability to bind to the desired antigen, e.g., α-cells or β-cells. It will also be understood by one of ordinary skill in the art that fusion proteins of the invention may be modified such that they vary in amino acid sequence from the original binding polypeptide from which they were derived and may have a certain percent identity to the starting sequence, e.g., it may be at least 60%, at least 70%, at least 75%, at least 80%, at least 85%, at least 90%, at least 95% or at least 99% identical to the starting sequence.

In another embodiment, the proteins of the invention are purified, or substantially pure.

The present invention is further directed to isolated polypeptides which make up the fusion proteins of the invention, and polynucleotides encoding such polypeptides. A polypeptide or amino acid sequence “derived from” a designated protein refers to the origin of the polypeptide having a certain amino acid sequence. In certain cases, the polypeptide or amino acid sequence which is derived from a particular starting polypeptide or amino acid sequence has an amino acid sequence that is essentially identical to that of the starting sequence, or a portion thereof, wherein the portion consists of at least 10-20 amino acids, at least 20-30 amino acids, at least 30-50 amino acids, or which is otherwise identifiable to one of ordinary skill in the art as having its origin in the starting sequence.

In one embodiment, the invention encompasses fusion proteins, or fragments, variants, analogs, homologs, modifications or derivatives thereof which retain the binding specificity of the fusion proteins, conjugated to a diagnostic agent. The fusion proteins can be used diagnostically to, for example, monitor the development or progression of diabetes in a subject as part of a clinical testing procedure to, e.g., determine the efficacy of a given treatment and/or prevention regimen. Detection can be facilitated by coupling the fusion protein of the invention to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions; such substances are well-known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, .beta.-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

Other suitable imaging agents for use with the fusion proteins of the present invention include, without limitation, ¹¹C, ¹⁸F, ¹⁸O, ¹³N, ⁷⁶Br and ¹²⁴I as radioligands in PET imaging or ⁸⁶Y, ^{99m}Tc, ¹¹¹In, ¹²³I, and ²⁰¹Tl as radioligands in SPECT imaging.

The person skilled in the art will appreciate that many imaging methods are known and can be used with the fusion proteins of the present invention. For example, presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), SPECT, bioluminescence, fluorescence and sonography. The person skilled in the art will be able to choose appropriate ligands, agents and methods using routine procedures. It will also be understood in the art that the size of the subject and the imaging system used will determine the quantity of imaging moiety needed to produce diagnostic images.

In a specific embodiment, the fusion protein is labeled with a radioisotope and is detected in the patient using a radiation responsive surgical instrument (see e.g. Thurston et al., U.S. Pat. No. 5,441,050). In another embodiment, the fusion protein is labeled with a fluorescent compound and is detected in the patient using a fluorescence responsive scanning instrument. In another embodiment, the fusion protein is labeled with a positron emitting metal and is detected in the patent using positron emission-tomography. In yet another embodiment, the fusion protein is labeled with a paramagnetic label and is detected in a patient using magnetic resonance imaging (MRI).

Techniques for conjugating various moieties to a fusion protein of the invention are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982).

With respect to the use of radiolabeled conjugates in conjunction with the fusion protein of the present invention, it should be understood that the fusion protein may be directly labeled (such as through iodination) or may be labeled indirectly through the use of a chelating agent. As used herein, the phrases “indirect labeling” and “indirect labeling approach” both mean that a chelating agent is covalently attached to a binding molecule and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Radionuclides for indirect labeling include ¹¹¹In and ⁹⁰Y.

As used herein, the phrases “direct labeling” and “direct labeling approach” both mean that a radionuclide is covalently attached directly to a polypeptide (typically via an amino acid residue). More specifically, these linking technologies include random labeling and site-directed labeling. In the latter case, the labeling is directed at specific sites on the polypeptide, such as the N-linked sugar residues present only on the Fc portion of the conjugates. Further, various direct labeling techniques and protocols are compatible with the instant invention. For example, Technetium-99 labeled polypeptides may be prepared by ligand exchange processes, by reducing pertechnate (TcO₄⁻) with stannous ion solution, chelating the reduced technetium onto a Sephadex column and applying the binding polypeptides to this column, or by batch labeling techniques, e.g. by incubating pertechnate, a reducing agent such as SnCl₂, a buffer solution such as a sodium-potassium phthalate-solution, and the antibodies. Preferred radionuclides for directly labeling antibodies are well known in the art.

It will be appreciated that, in accordance with the teachings herein, the fusion proteins may be conjugated to many different radiolabels for diagnostic and imaging purposes. A variety of radionuclides are applicable to the present invention and those skilled in the can readily determine which radionuclide is most appropriate under various circumstances. Fusion proteins can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

It is known in the art that after phage selection, as described herein, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)₂ fragments can be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). In an embodiment, such single-chain Fvs and antibodies are used in the fusion proteins of the invention.

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, which are incorporated herein by reference in their entireties. Humanized antibodies are antibody molecules from non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment or diagnosis of human patients. Human antibodies can be made by a variety of methods known in the art including phage display methods described herein using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

In an embodiment, the fusion proteins or analogs, homologs, fragments or variants thereof provided herein comprise SCAs which are humanized or fully human.

In another embodiment, any antigen-binding polypeptide which binds specifically to the same α-cell or β-cell specific epitope as the SCAs provided herein, is encompassed for use in the fusion proteins of the invention.

### Pharmaceutical Compositions and Methods of Administration

Pharmaceutical compositions encompassing the fusion proteins of the invention are also encompassed herein. The fusion proteins of the present invention can be administered to a subject in a conventional dosage form prepared by combining the fusion protein of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables.

Methods of preparing and administering fusion proteins or antigen-specific analogs, homologs, fragments or variants thereof to a subject are well-known in the art or are readily determined by those skilled in the art. The route of administration of the fusion proteins of the invention may be, for example, oral, parenteral, by inhalation or topical. The term parenteral as used herein includes, e.g., intravenous, intraarterial, intraperitoneal, intramuscular, subcutaneous, rectal or vaginal administration.

Usually, a suitable pharmaceutical composition for injection may comprise a buffer (e.g. acetate, phosphate or citrate buffer), a surfactant (e.g. polysorbate), optionally a stabilizer agent (e.g. human albumin), etc. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating a fusion protein of the invention (by itself or in combination with other active agents) in the required amount in an appropriate solvent with one or a combination of ingredients, as required and easily determined by a person of skill in the art, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art.

Further, the preparations may be packaged and sold in the form of a kit. Such articles of manufacture will preferably have labels or package inserts providing instructions for use and may have additional components required for the use of the preparations.

Those skilled in the art will appreciate that effective doses of the fusion proteins and compositions of the present invention, e.g. for preventing or treating diabetes or for in vivo imaging of α- or β-cells of the pancreas as described herein, vary depending upon many different factors, including means of administration, characteristics or physiological state of the subject (such as state of health), other medications being administered, whether the treatment is diagnostic, prognostic, prophylactic or therapeutic, and so on. The dosage may be determined using routine methods known to those of skill in the art in order to optimize safety and efficacy.

Clearly, the amount of the fusion peptide to be administered will also depend on the subject to which it is to be administered. In the case where the subject is a human, the amount of the peptide to be administered will depend on a number of factors including the age of the patient, the severity of the condition and the past medical history of the patient and always lies within the sound discretion of the administering physician. Generally, the total daily dose of the fusion proteins of the invention administered to a human or other mammal in single or in divided doses can be in amounts, for example, of from 0.1 mg/Kg/day to 30 mg/Kg/day of the peptide, preferably from 0.1 mg/Kg/day to 20 mg/Kg/day of the peptide, more preferably from 2 mg/Kg/day to 10 mg/Kg/day of the peptide, in single or multiple doses. Single dose compositions may contain such amounts or submultiples thereof to make up the daily dose. In a particular embodiment, the administration route is intravenous.

The peptides of the present invention may also be administered as a component of a pharmaceutically administrable composition. In other words, the peptide may be present in a formulation for administration to a subject in need thereof. The inventive peptide may be the sole active ingredient for e.g. NF-kB pathway inhibition or treatment of diabetes. Alternatively, the composition may also contain one or more additional compounds that may be used to treat the same or related conditions. In addition, the peptide of the present invention may be in a composition that contains one or more compounds that are useful for treatment of a disorder not caused by the NF-kB pathway.

It should be understood that fusion proteins of the invention can optionally be administered in combination with other agents that are effective in treating the disorder or condition in need of treatment. In keeping with the scope of the present disclosure, α- or β-cell specific fusion proteins of the invention may be used with other therapeutic or prophylactic agents. The agents may be administered together or separately. Examples of such therapeutic or prophylactic agents include, without limitation, anti-apoptotic substances such as the Nemo-Binding Domain and compounds that induce proliferation such as CDK-6, CDK-4 and Cyclin D1. The fusion proteins according to the invention may also be used in conjunction with other active agents, particularly for the treatment and/or prevention of the diseases and conditions mentioned above. For example, other therapeutically or prophylactically active agents which are suitable for such combinations include antidiabetic agents such as metformin, sulphonylureas (e.g. glibenclamide, tolbutamide, glimepiride), nateglinide, repaglinide, thiazolidinediones (e.g. rosiglitazone, pioglitazone), PPAR-gamma-agonists (e.g. C1262570) and antagonists, PPAR-gamma/alpha modulators (e.g. KRP 297), alpha-glucosidase inhibitors (e.g. acarbose, voglibose), DPPIV inhibitors (e.g. LAF237, MK-431), alpha2-antagonists, agents for lowering blood sugar, cholesterol-absorption inhibitors, HMGCoA reductase inhibitors (such as a statin), insulin and insulin analogues, GLP-1 and GLP-1 analogues (e.g. exendin-4) or amylin.

### Examples

The present invention will be more readily understood by referring to the following examples, which are provided to illustrate the invention and are not to be construed as limiting the scope thereof in any manner.

Generation of SCAs Binding Selectively to β- or α-cells

For the purpose of identifying and generating agents specifically binding to pancreatic islets in vivo, a phage library was screened for SCAs on rat islets using two different approaches: 1) Islets were isolated from rats after intravenous injection of the library for a circulation time of 5 minutes; and 2) Rat islets were isolated and the library was panned in the isolated islets in vitro. Five rounds of selection were carried out and demonstrated a marked increase in the phage transducing units (TU) per islet over successive rounds of panning, representing a 700- and 500-fold enrichment within the first and the second approach, respectively. Subsequently, the DNA encoding the corresponding phage-displayed SCAs was sequenced. The first approach supplied five islet-specific phage clones (ISPCs), ISPC1 to 4 and 7. The second approach generated two ISPCs, ISPC5 and 6 (FIG. 1).

### Specific Binding to Human Islets In Situ and Exclusion of Toxicity

The selective binding of the ISPCs to rat islets in vivo was determined by the harvest of the pancreas and control organs after intravenous administration of the ISPCs for a circulation time of 2 hours. In rats that received ISPC1, phage immunostaining was clearly identifiable in the cytoplasm of the islets and the signal overlapped with insulin expression, whereas no co-staining with glucagon was detectable, suggesting that the ISPC1 was exclusively taken up by β-cells (see WO/2010/096930). The same staining patterns were observed for ISPC 2, 5, 6 and 7 (see WO/2010/096930). In contrast, in animals that received ISPC3 or 4 (see WO/2010/096930), phage immunostaining was clearly identifiable and overlapped exclusively with glucagon expression, suggesting that ISPC3 and 4 were taken up by α-cells. Importantly, a control ISPC without insert was undetectable in the islet (see WO/2010/096930). Moreover, none of the β-cell specific ISPC 1, 2, 5 to 7 and the α-cell specific ISPC-3 and 4 exhibited any binding to control organs, such as liver, kidney, spleen, heart, lung and exocrine cells (see WO/2010/096930).

Next, all ISPCs were produced as soluble SCAs containing a c-Myc tag and a His₆ tag in small scale cultures, purified by metal affinity chromatography and then intravenously applied to rats. Highly selective uptake into the cytoplasm of β-cells was confirmed for SCA B1, 2, 5, 6 and 7, whereas SCA A1 and 4 (see WO/2010/096930) were accumulated selectively in the α-cells of the islet (see WO/2010/096930). In addition, none of SCA B1 to B5 and SCA A1 and 2 exhibited any binding to control organs, such as liver, kidney, spleen, heart, lung and exocrine cells.

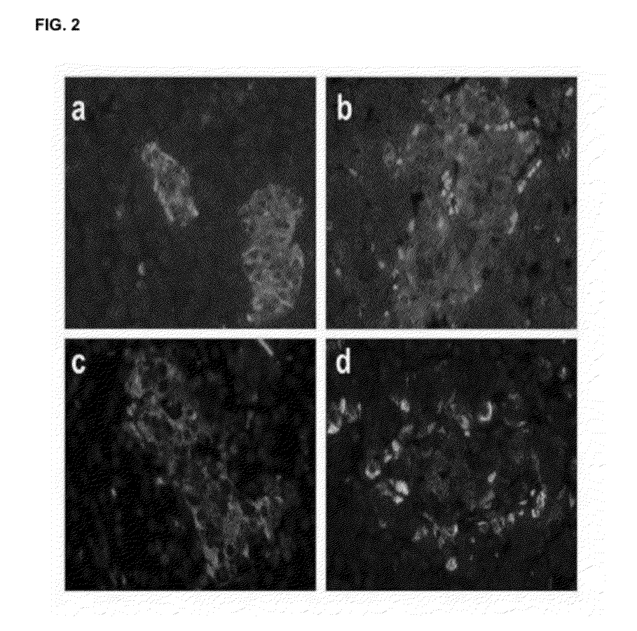

We have shown that the generated SCA detect a protein or receptor that is expressed selectively in human pancreatic β- or α-cells. The selectivity of the SCAs was demonstrated by exposure to human pancreatic tissue slides, where SCAs were detected using a monoclonal anti-cMyc antibody, and co-staining with specific anti-insulin- and anti-glucagon-antibodies was used to identify the cell type. Staining for β-cell specific SCA B1 was clearly identified and restricted to the islets and co-localized with insulin expression, whereas none was detectable in α- or exocrine cells (FIGS. 2 a,b). In contrast, the α-cell specific SCA A1 overlapped with glucagon staining, but not with insulin expressing- or exocrine-cells (FIGS. 2 c,d). These data strongly suggest that the SCA bind to a protein/receptor that is selectively expressed in human islets.

Thus, SCA with highly specific binding to pancreatic β-cells or a-cells in vivo are provided. SCAs have been generated which are internalized into the insulin producing β-cells of rat pancreas in vivo, and bind to human β-cells in situ. These SCAs have been linked to the endoplasmatic reticulum and the insulin secretory granule membrane by transmission electron microscopy. Similarly, we generated two SCAs that were selectively taken up by the glucagon-producing α-cells of rats in vivo, exhibited highly specific binding to human α-cells in situ and were linked as well to the endoplasmatic reticulum and the glucagon secretory granule. Importantly, no binding to exocrine cells or other tested tissues was detected for any of the generated SCAs in vivo. These results, together with the unexpected specificity ratios for target- vs. non-target-cells determined on cell lines in vitro, indicate strongly the selectivity of the generated SCAs to either β- or α-cells.

Without wishing to be bound by theory, it is likely that the mechanism of binding and cellular uptake within the short time frame of minutes and at the observed high quantities of more than 650,000 SCA per cell, to either β- or α-cells, is a membrane protein/receptor driven process.

In summary, it has been established that SCA B1 and its homologs and SCA A1 and its homologs are highly selective for binding to β- or α-cells in vivo.

SCA B1-NBD Peptide is 8-cell Specific

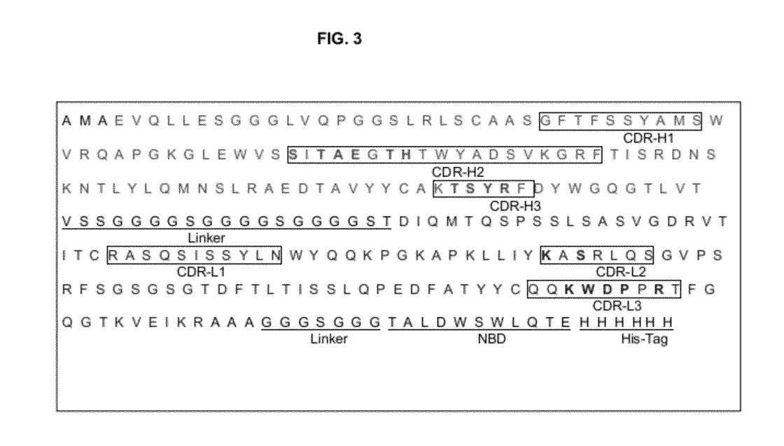

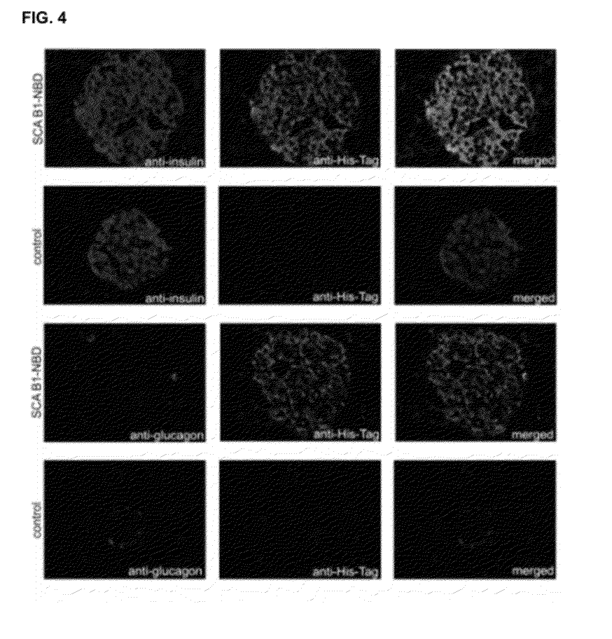

In order to generate a novel agent that protects β-cells in vivo against the detrimental effects of cytokines, the SCA B1-NBD fusion protein was cloned in the pIRES-EGFP, expressed in BL21 bacteria and purified by metal affinity chromatography (the amino acid sequence is given in FIG. 3). Subsequently, the SCA B1-NBD peptide was intravenously injected in non-diabetic CD rats and the pancreas harvested and prepared for immunohistochemical analyses. By these means, we confirmed β-cell specific accumulation of SCA B1-NBD peptide in the pancreas (FIG. 4). Importantly, the SCA B1-NBD peptide was not detectable in any of the control tissues (liver, spleen, kidney, brain; data not shown). Of note, these results are comparable to the in vivo biodistribution of the β-cell specific SCAs (SCA B1-B5), as described above and reported previously (Ueberberg, S. et al., Diabetologia. 53(7):1384-94, 2010; Ueberberg, S. et al., Diabetes 58(10):2324-34, 2009; WO/2010/096930).

### SCA B1-NBD Peptide Protects Islet Cells Against IL-1β In Vitro

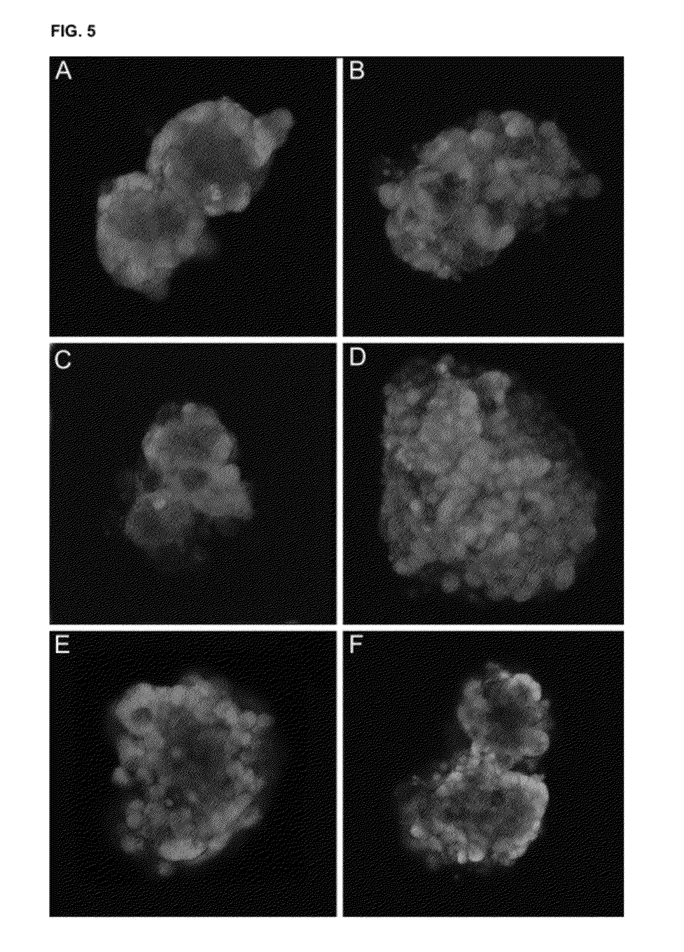

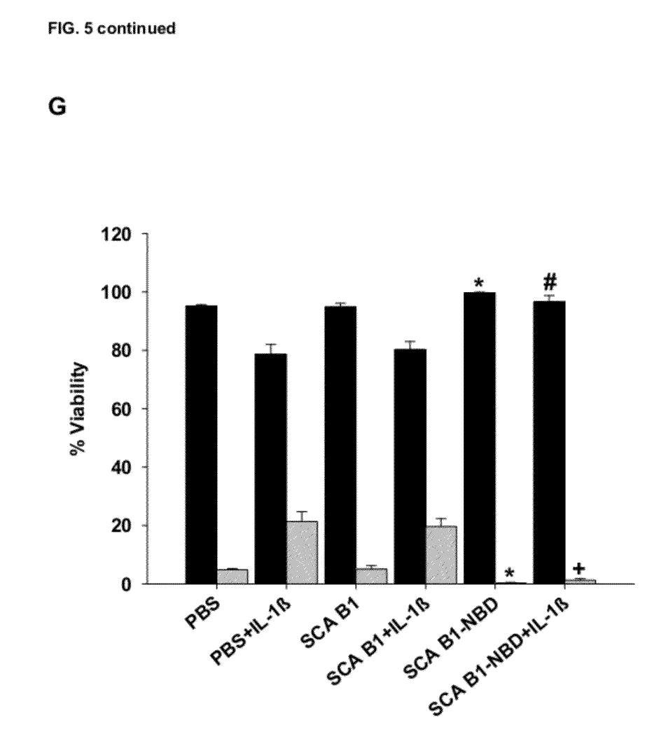

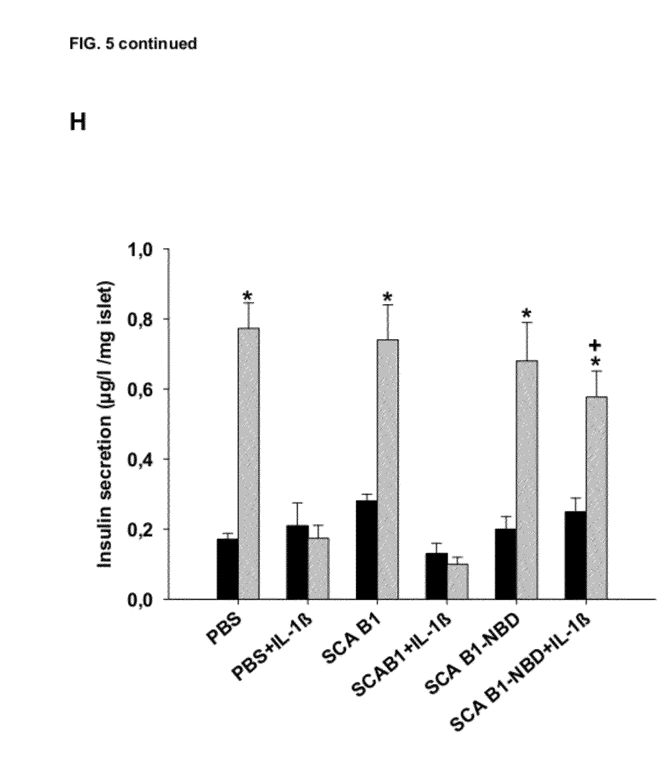

In order to determine whether the SCA B1-NBD peptide protects islet cells against the detrimental effects of IL-1β in vitro, we injected the SCA B1-NBD peptide intravenously in normal CD rats followed by islet isolation. After an overnight culture, we incubated the islets either in the presence or absence of IL-1β for 24 h and analyzed viability and glucose-induced insulin secretion of the islets, respectively. By these means, we showed that in vivo delivery of SCA B1-NBD peptide significantly improved islet viability compared to islets of SCA B1 or PBS treated rats (FIG. 5). Moreover, we demonstrated that islets of SCA B1-NBD treated rats cultured in the presence of IL-1β showed significantly greater viability compared to islets of rats treated either with SCA B1 or PBS (FIG. 5). To confirm that improved cell survival by SCA B1-NBD peptide treatment resulted in preserved islet function, the ability of the islets to respond to a glucose challenge was examined. By these means, we could clearly show that SCA B1-NBD treatment was able to prevent IL-1β-induced impairment of glucose-induced insulin secretion, whereas in the control groups the glucose-induced insulin secretion was completely absent (FIG. 5). Taken together, in vivo treatment of rats with SCA B1-NBD peptide protect islet cells against the detrimental effects of IL-1β in vitro.

SCA B1-NBD Peptide Inhibits NF-KB Activation and Protects β-cells Against Diabetogenic Agents In Vivo

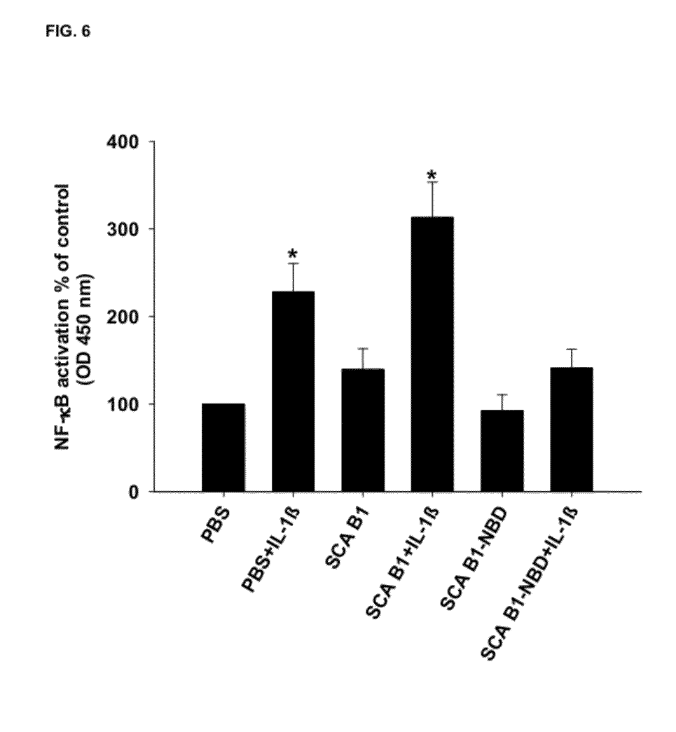

In order to demonstrate that in vivo delivery of the SCA B1-NBD inhibited NF-κB activation, we analysed NF-κB binding activity in these islets, using a NF-κB p65 transcription factor assay. As shown in FIG. 6, NF-κB activity was significantly increased in islets of rats treated with SCA B1 or PBS following treatment with IL-1β. However, in the SCA B1-NBD peptide treated islets exposed to IL-1β, the NF-κB level was similar to control islets. These results demonstrate that selective in vivo delivery of the NBD peptide into β-cells blocked IL-1β-mediated induction of NF-κB binding activity.

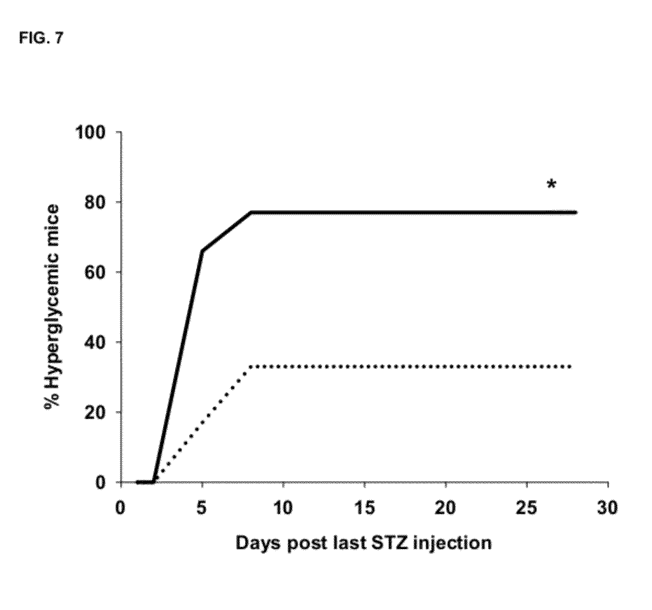

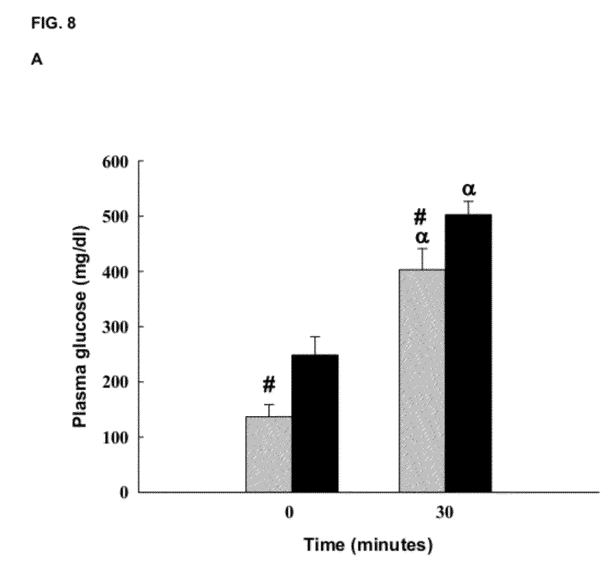

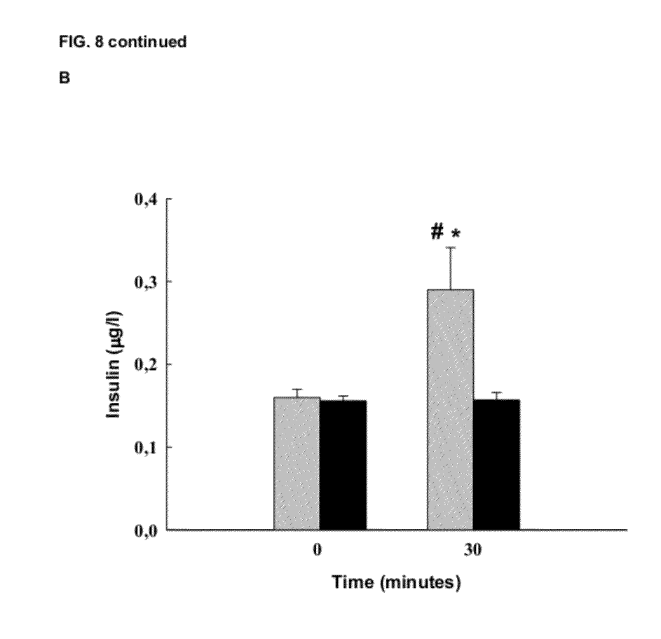

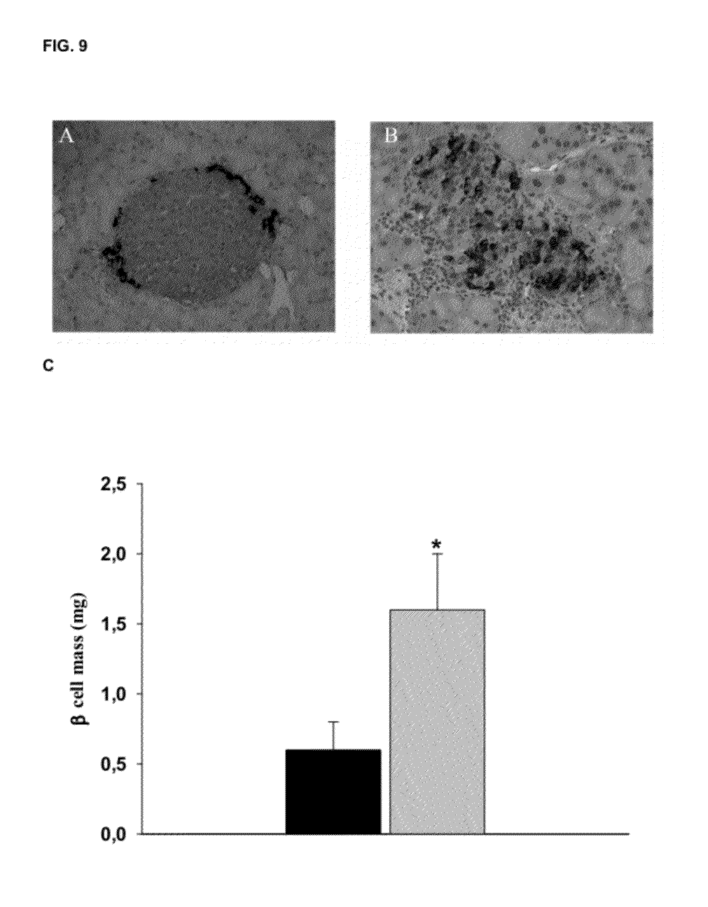

To test the ability of the SCA B1-NBD peptide to protect β-cells against diabetogenic agents in vivo, CD mice were treated by intravenous injection of the SCA B1-NBD peptide on a daily basis prior to multiple low-dose STZ-treatment (MLDS), Subsequently, we monitored for the appearance of diabetes. Our results show a striking resistance to the development of diabetes after MLDS in SCA B1-NBD treated as compared with PBS-treated mice. In fact, 7 of 9 mice from the PBS-treated group gradually developed diabetes 5-10 days after the last injection of STZ as opposed to only 2 of 6 SCA B1-NBD treated mice (FIG. 7). Furthermore, we analyzed plasma concentration of glucose and insulin during an IPGTT and β-cell mass by morphometry 28 days after MLDS, respectively. The experiments revealed that both fasting and post-challenge glucose concentrations were significantly higher and post-challenge insulin levels significantly lower in the PBS-treated compared to SCA B1-NBD treated mice (FIG. 8). Finally, the results correlated with reduced β-cell mass detected in the pancreata from PBS-treated vs. SCA B1-NBD treated mice (FIG. 9).

### Experimental Methods

### Animal Models, Human Tissue Samples, Phage Library, Cell Lines

Female 6-week old CD-rats were purchased from Charles River Laboratories. Mild or severe diabetic animals were established by intraperitoneal injection with 30 or 60 mg/kg STZ two weeks prior to the experiments. Animals with plasma glucose levels of more than 350 mg/dl in four consecutive measurements were used in the experiments approved by the Landesamt für Naturschutz (No. 50.10.32.08.037). The use of human pancreatic tissue samples, obtained from non-diabetic patients undergoing partial pancreatectomy, was approved by the ethics committee of the Ruhr-University Bochum (No. 2528, amendment 3).

The recombinant phage-library Tomlinson I is constructed in the pIT2 vector (derived from pHEN1), consists of about 1.4×10⁸ different human single chain variable fragments and was provided by MRC Genservice (Cambridge, UK). The library is based on a single human framework for VH (V3-23/DP-47 and JH4b) and V (O12/O2/DPK9 and J1) with diversified (DVT) side chains incorporated in complementary determining region 2 and complementary determining region 3 at positions in the antigen binding site that make contacts to antigen in known structures and are highly diverse in the mature repertoire.

Cell lines used in the studies were rat β-cell line INS-1, the murine α-cell line α-TC1 and the rat exocrine cell line AR42J.

### Phage Library Screening

For the in vivo approach a rat was injected through the jugular vein with 10¹² phage TU for a circulation time of 5 minutes, followed by islet isolation using collagenase digestion. Subsequently islet purification was achieved using a discontinuous three-phase Ficoll density gradient. In the in vitro approach rat islets were directly isolated, and incubated with 10¹² TU for 1 h at 37° C. with gentle shaking.

Following both screening approaches, the islets were washed twice in 1 ml HBSS, once in 1-ml wash with 0.1 N HCl-Glycine (pH 2.2). Subsequently, the cells were lysed by the addition of 1 ml hypotonic solution (30 mmol/l Tris-HCl, pH 8.0), followed by a single freeze-thaw cycle. Phages from the output fraction were amplified according to standard protocols for use in the next round of panning. Aliquots of the initial input and output samples were titered, and the output-to-input phage ratio based on colony-forming units was determined to monitor the progress of each round of library panning.

### Generation and Purification of SCA

HB2151-bacteria (OD₆₀₀=0.4) were infected with the ISPC of interest and grown overnight at 37° C. on 2TY plates containing 100 μg/ml ampicillin and 1% glucose. On the following day individual colonies were picked and grown for 12 h in 10 ml 2TY containing 100 μg/ml ampicillin and 1% glucose (shaking at 220 rpm at 37° C.). Subsequently, the 10 ml culture was added to 2 L of 2TY medium (0.1% glucose and 100 μg/ml ampicillin) and grown at 37° C. until an OD₆₀₀=0.6 was obtained. Isopropyl β-D-thiogalactoside (IPTG; AppliChem GmbH, Darmstadt, Germany) (final concentration 1 mM) was added to the culture to induce SCA expression. The culture was grown for 4 h at 30° C. (shaking at 220 rpm), followed by centrifugation at 6000 g for 15 min at 4° C. The pellet was re-suspended in 50 ml PBS and 1 mM Phenylmethylsulfonylfluoride (PMSF; Roche Applied Science, Mannheim, Germany) and incubated for 1 h on ice, vortexed intermittently, followed by centrifugation for 1 h at 11600 g at 4° C. The supernatant containing the SCA was purified by immobilized metal affinity chromatography on a nickel column (Nunc ProPur®, Nunc GmbH, Germany) according to the manufacturer's protocol, except for more intensive washing (3-4 times). Imidazole (80 mM) was used to elute the SCA from the column. The purified sample was then dialyzed overnight in PBS and purity was checked by SDS gel electrophoresis. The SCA concentration was determined using the BCA protein assay kit (Pierce, Rockford, USA).

### Cell Culture

INS-1 cells (kind gift from C. Wollheim, Geneva) were grown in RPMI 1640 (Gibco BRL, Grand Island, N.Y.) supplemented with 10% (v/v) fetal bovine serum (FBS), 2 mM glutamine (all from Gibco BRL), 1 mM pyruvate and 50 μM β-mercaptoethanol (Sigma Chemical, St. Louis, Mo.), AR42J (ATCC, Manassas, Va.) cells were grown in Dulbecco's modified Eagle's medium (DMEM) media (Gibco BRL) containing 10% FBS. Cells were detached with trypsin (0.05% trypsin, 0.53 mM EDTA-4Na, Gibco BRL) 2 hours prior to conducting the screening assays as follows. Culture media bathing the cells was aspirated and the cells were washed with phosphate-buffered saline (PBS) to remove residual media/serum. Trypsin/EDTA (2.5 mL) was added to each T-75 and the flasks were incubated for 3 minutes after placing in a CO₂ incubator maintained at 37° C. Cell culture media (RPMI with 10% FBS) was added and the cell suspension was transferred to a 50 mL conical centrifuge tube, and centrifuged at 1000×g for 3 minutes. The supernatant was aspirated; the cells were resuspended in culture media to achieve approximately 1 million cells/ml, and then stored in a CO₂ incubator at 37° C. until the binding studies were conducted.

### Immunohistochemistry

Staining of formalin-fixed, paraffin-embedded rat and human tissue sections (5 μm) were performed as follows: Sections were deparaffinized using Xylol twice for 10 min and followed by EtOH three times for 5 min and Aqua dest. for another 5 min. Afterwards, the sections were permeabilized by heating in the microwave in antigen unmasking solution pH 6 and cooling down for 45 min. Blocking was done for 1 h at 24° C. with PBS containing 2% BSA. Primary and secondary antibodies were diluted in PBS with 2% BSA. Primary antibodies were incubated at 4° C. overnight, except for insulin and glucagon for which the incubation period was 1 h at 37° C. Secondary antibodies were incubated for 30 min at 24° C. and the same holds true for the Cy2- and Cy3-conjugated streptavidin reagents. The following primary antibodies and dilutions were used: SCA B1 and SCA A1, 1:200; monoclonal mouse anti-c Myc antibody, 1:200 (Cell Signaling, #2276); polyclonal guinea pig anti-swine-insulin antibody, 1:400 (Dako, #A0564); and monoclonal mouse anti-glucagon antibody, 1:200 (Affinity BioReagents, #MA1-20210). Secondary antibodies were monoclonal mouse anti-c Myc antibody, 1:200 (Cell Signaling, #2276); biotinylated anti-rabbit IgG and biotinylated anti-mouse IgG, 1:200 (Linaris, #BA-1000, #BA-2001); Cy3-conjugated goat anti-mouse IgG, 1:200 (Jackson ImmunoResearch Laboratories, #115-165-044); Cy3-conjugated goat anti-guinea pig IgG, 1:800 (Jackson ImmunoResearch Laboratories, #106-165-003). Third reagents were Cy2-conjugated streptavidin, 1:200 (Jackson ImmunoResearch Laboratories, #016-220-084). Tissue slides were analyzed using a Zeiss Axioplan microscope.

### Electron Microscopy

Rats were transcardially perfused with PBS and 2.5% glutaraldehyde to fix the pancreas before extraction, postfixed in 2.5% glutaraldehyde, rinsed with PBS, postfixed in 1% osmiumtetroxide and dehydrated in ascending concentrations of ethanol and propylenoxide and embedded in durcupan. Ultrathin sections were stained with ultrostain 1 and 2 using the ultrostain. Residual aldehyde groups were inactivated with 0.05M lysine in PBS buffer. Block Step: 5% BSA and 0.1% CWSFs gelatin supplemented with 5% serum, followed by incubation for 1 h with monoclonal mouse anti-c Myc antibody (1:200; Cell Signaling, #2276) and a biotinylated IgG (1:200, Vector Laboratories). For gold labelling ultra small gold (Aurion, Wageningen, Netherlands) and silver enhancement (Aurion, Wageningen, Netherlands) was used. Sections were analyzed on a ZEISS 109 transmission electron microscope.

### In Vitro Screening Assay

Polypropylene test tubes (12×75 mm) containing cells (0.25×10⁶) in 100 μl of RPMI1640 (prepared as described above) were placed in an incubator (5% CO2/37° C.) for 30 min. The incubator door was opened, and the addition of the radiolabeled SCA was accomplished using an Eppendorf Repeater pipet and subsequently the cells were further incubated for 30 min (or as noted). Accumulation of radiolabel was determined by separating the cell-associated radioactivity (CAR) from the free radioactivity by transferring the cell suspension to a 0.4-ml centrifuge tube (USA Scientific, Ocala, Fla.) containing a layer of oil consisting of 1:90.8 volume to volume ratio of n-dodecane: bromo-dodecane (yields 1.017 g/ml; Sigma-Aldrich), and spinning for 8 seconds in a Beckman E centrifuge (maximum speed=12,535 g Beckman Coulter Inc., Fullerton, Calif.). The tubes were placed briefly in liquid nitrogen, cut through the radioactive-free oil layer with a razor blade, and the bottom portion of the tube containing the cell pellet was placed into a 12×75 mm polypropylene test tube and counted in a gamma counter. Retention was determined similarly except that after incubating the cells for 30 min in the presence of the radiolabel, cells were washed twice with RPMI1640 and further incubated in radiolabel-free media prior to spinning the cells through the oil layer. In order to account for the cell type specific cell volume the CAR (cpm/cell) was normalized with the average cell volume (fl), determined in each experiment with a CASY®. To evaluate specificity of binding, SCAs were preincubated with selected unlabelled SCAs (20 μg) for competition assays.

### Materials and Methods

### Generation of SCA B1-NBD Fusion Protein.

The NBD peptide (TALDWSWLQTE) was C-terminal fused to the SCA B1 with a flexible Glycine-Serine linker and the SCA B1-NBD construct was cloned into the multiple cloning site of pIRES-EGFP and the SCA B1-NBD-IRES-EGFP fusion protein was expressed in BL21 bacteria. The fusion protein was then transformed into BL21 E. coli cells and plated on LB-plates (50 μg/ml kanamycin) overnight at 37° C. A single colony was further grown in LB-medium (50 μg/ml kanamycin) at 37° C. until the OD₆₀₀ reached 0.9. Isopropyl β-D-thiogalctoside (1 mM final concentration) was added to induce SCA B1-NBD peptide expression and finally the SCA B1-NBD peptide was purified from the supernatant of this culture by metal affinity chromatography (Nunc GmbH, Langenselbold, Germany). The purity of the sample was checked by SDS gel electrophoresis and western blotting.

### In Vivo Biodistribution of SCA B1-NBD Peptide.

Female non-diabetic CD® rats were purchased from Charles River (Sulzfeld, Germany). The rats were injected intravenously with either SCA B1-NBD (100 μg protein in 100 μl phosphate buffered saline (PBS); n=5) or PBS (100 μl; n=5). Rats were killed 2 hours (h) after injection and organs (as stated) were harvested and fixed with formalin. Staining of paraffin-embedded rat tissue sections (5 μm) was performed as follows: Sections were deparaffinised and subsequently permeabilised by heating in the microwave oven in antigen-unmasking solution pH 6 and cooling for 45 min. Blocking was done for 1 h at 24° C. with PBS containing 2% (wt/vol) bovine serum albumin (BSA, Sigma Aldrich, Steinheim, Germany). Primary and secondary antibodies were diluted in PBS with 2% BSA, Primary antibodies were incubated at 4° C. overnight, except for anti-insulin and anti-glucagon antibodies with an incubation period of 1 h at 37° C. Secondary antibodies were incubated for 30 min at 24° C. as well as Cy2- and Cy3-conjugated streptavidin reagents. The following primary antibodies and dilutions were used: sheep anti-His-tag, 1:200 (Antikörper-online.de); guinea pig anti-swine-insulin, 1:400 (Dako, Carpinteria, Calif., USA) and mouse anti-glucagon, 1:200 (Thermo Scientific, Rockford, Ill., USA). Secondary antibodies were biotinylated anti-sheep IgG 1:100 (Linaris GmbH, Wertheim-Bettingen, Germany); Cy3-conjugated goat anti-mouse IgG, 1:200 (Jackson Immuno Research Europe Ltd., Suffolk, UK); and Cy3-conjugated goat anti-guinea pig IgG, 1:800 (Jackson Immuno Research Europe Ltd., Suffolk, UK). The tertiary reagent was Cy2-conjugated streptavidin, 1:200 (Jackson Immuno Research Europe Ltd., Suffolk, UK). Tissue slides were analysed using a Zeiss Axioplan microscope.

### In Vivo Delivery of SCA B1-NBD Peptide and Islet Isolation.

Female non-diabetic CD® rats were injected intravenously either with SCA B1-NBD, SCA B1 (100 μg protein in 100 μl PBS) or PBS (100 μl). Two hours after injection isolation of islets was performed (Schneider S, et al., Diabetes 54(3):687-93, 2005). Briefly, CD® rats were anaesthetised by intraperitoneal pentobarbital administration (60 mg/kg). Then a midline abdominal incision was performed, the pancreas was exposed and injected via the pancreatic duct with Hank's balanced salt solution (HBSS; Biochrom KG, Berlin, Germany) containing 0.5 mg/ml collagenase (Serva PanPlus, Heidelberg, Germany). After sacrificing the animal, the pancreatic tissue was surgically removed and incubated for 7 min at 37° C. in the collagenase solution. Mechanical disruption of the digested pancreatic tissue was achieved by further incubation in collagenase solution at 37° C. for 7 min, interrupted every 80 seconds by shaking for 15 seconds. Digestion was stopped by addition of 4° C.-cold HBSS plus 10% BSA. Islet purification was achieved using a discontinuous three-phase Ficoll density gradient (densities: 1.090, 1.077 and 1.040). Subsequently, the islets were cultured overnight in RPMI 1640 medium (Biochrom KG, Berlin, Germany) supplemented with 100 mg/dl glucose, 10% BSA and antibiotics (100 units/ml penicillin, 100 μg/ml streptomycin; GibcoBRL, Paisley, Scotland) at 37° C./5% CO₂.

### Experimental Procedures

### Assessment of Islet Viability and Glucose-induced Insulin Secretion Following In Vivo Delivery of SCA B1-NBD and Exposure to IL-1β In Vitro

To determine the protective effects of NF-κB inhibition in the presence and absence of IL-1β treatment, viability and glucose-stimulated insulin release was analyzed. In vivo delivered islets with either SCA B1-NBD, SCA B1 or control islets (PBS injected only) were incubated in the presence or absence of recombinant rat IL-1β (2 ng/ml; R&D Systems, Minneapolis, USA) for a period of 24 h prior to viability assessment or insulin release studies. The IL-1β containing medium was then removed and the islets were washed twice in RPMI 1640 medium without BSA. For viability assessment, the islets were incubated in the presence of 16.3 μg/ml Calcein AM (Invitrogen, Karlsruhe, Germany) and 10 μg/ml propidium iodide (Invitrogen, Karlsruhe, Germany) for 30 min at 37° C. as described previously (Rehman K K, et al., J. Biol. Chem. 278(11):9862-8, 2003). Subsequently, pictures were captured by a two-photon confocal microscope and percent viability was analyzed using MetaMorph™ software package version 4.6r9 (Universal Imaging Corp., Downingtown, Pa.). Percentage of viable cell aggregates over the total was determined by scoring green versus red fluorescence in at least 25-30 islet cell aggregates.

For insulin secretion studies, 50 islets in triplicate were handpicked and transferred into a culture-insert (membrane pore diameter 12 μm; Millicell PCF, Millipore, Schwalbach, Germany). The insert was put into a well of a 24-well culture-plate (Peske, Aindling-Arnhofen, Germany) and islets were incubated for 1.5 h at 37° C./5% CO₂ in RPMI-medium under low glucose concentrations (100 mg/dl). The RPMI-medium was removed and stored, and replaced with RPMI-medium containing high glucose (350 mg/dl) and incubated for another period of 1.5 h at 37° C./5% CO₂. Subsequently, insulin was determined (see below). To normalize insulin secretion, the islet protein content of each culture insert was determined.

### NF-κB p65 Transcription Factor Assay Following In Vivo Delivery of SCA B1-NBD and Exposure to IL-1β In Vitro.

To examine the protective effect of SCA B1-NBD peptide from IL-1β exposure on NF-κB activity in vitro, isolated islets of SCA B1-NBD, SCA B1 and PBS treated rats were analyzed. Briefly, islets of the respective groups were cultured in the presence or absence of recombinant rat IL-1β (2 ng/ml) for a period of 2 h, subsequently washed twice with cold PBS and stored at −20° C., until whole cell extracts were prepared. A total of 10 μg of cellular protein from each group was analyzed for p65 binding activity, using the enzyme-linked immunosorbent assay-based Trans-AM™ NF-κB p65 transcription factor assay kit (ActiveMotif, Carlsbad, Calif., USA). NF-κB binding activity was measured at 450 nm and the OD reading normalized to protein content.

### Multiple Low Dose Streptozotocin Diabetes Model.

CD®-1 mice (6-weeks of age, 20-25 g) were purchased from Charles River (Sulzfeld, Germany). Mice (n=15) were treated by intraperitoneal injections of streptozotocin (STZ; 40 mg/kg body weight; Sigma Aldrich, Steinheim, Germany), dissolved in fresh citrate buffer (100 mM citrate, pH 4.5) on five consecutive days. Six of these mice were treated with SCA B1-NBD (50 μg protein in 50 μl PBS) on a daily basis by tail vein injection 2 h prior to STZ-administration, whereas control mice (n=9) were injected with PBS only. Blood glucose determination was performed on blood samples taken from tail vein at day 0 before any injection and as stated. Hyperglycemia was defined as a non-fasting blood glucose level >200 mg/dl in three sequential measurements. At day 28, groups of mice (as stated) were sacrificed for morphological examination of the pancreas.

### Morphometric Analysis in Mice.

For the determinations of pancreatic β-cell mass, three longitudinal sections (−50 μm apart) through the entire pancreas were taken, stained for insulin and imaged at ×100 magnification (×10 objective) using a Zeiss Axioplan microscope equipped with a motorized stage. A tile image of the entire tissue section was generated using the Mosaix tool of Zeiss Axiovision version 4.5 software. The fractional areas of the pancreas stained positive for insulin were digitally quantified using a color-based threshold using Axiovision software. Subsequently, the β-cell mass was determined according to the following formula: BCM per pancreas (mg)=beta-cell fractional area x pancreatic weight (mg).

For insulin staining, the sections were deparaffinised and permeabilised (see above) and incubated with the primary guinea pig antibody against insulin (1:400 (diluted in PBS with 2% BSA; Dako, Carpinteria, Calif., USA) overnight at 4° C. Insulin was detected by the use of the streptavidin-alkaline/phosphatase method (Dako, Carpinteria, Calif., USA) according to the manufacturer's protocol.

### Intraperitoneal Glucose Tolerance Test (IPGTT).

At day 28 IPGTTs were performed in fasting (12 hours), unanaesthetized mice. After baseline blood sampling (0 minutes), animals received an intraperitoneal injection of glucose (2 g per kg body weight), with glucose and insulin concentrations measured at t=30 minutes after glucose administration. Blood samples were taken from the tail vein.

### Assay Procedures.

Glucose concentrations were determined by means of the glucose-oxidase method with a clinical analyzer (Nova Biomedical, Rodermark, Germany). Mouse insulin was determined using an ultrasensitive mouse ELISA (Mercodia, Uppsala, Sweden). The determination of mouse insulin was performed in duplicate with 5 μl sample volume. The detection limit of the assay was ≦ 0.188 μg/l. In contrast, rat insulin was determined with a rat insulin ELISA (Mercodia, Uppsala, Sweden), performed in duplicate with 10 μl sample volume. The detection limit of this assay was 1.5 μg/l.

### Statistics.

Parametric comparisons of continuous data were calculated with Student's t test for unpaired data with unequal variance. Differences in the appearance of diabetes, comparing the percentage of diabetic mice in each group, were calculated by Fisher's exact test. All calculations have been performed with KaleidaGraph 4.0.3 for Macintosh Computers (Synergy Software, Reading, Pa. USA).

In summary, we report herein the successful generation of a SCA B1-NBD fusion protein that accumulates highly selectively in pancreatic β-cells in vivo. Importantly, treatment of rodents with SCA B1-NBD by a simple intravenous injection prevented IL-1β-induced impairment of viability and glucose-stimulated insulin secretion of rat islet cells in vitro, and induced nearly complete protection against MLDS-induced diabetes in mice in vivo. The SCA B1-NBD fusion protein was highly specific for β-cells in vivo after intravenous injection in rats, with no relevant binding to other islet cells, exocrine cells or other tissues. We have shown SCA B1-mediated in vivo delivery of the NBD peptide in rats, prior to islet isolation.

The NBD peptide is a selective inhibitor of NF-κB activation. We further report herein that the SCA B1-NBD fusion is able to block IL-1β-mediated induction of NF-κB as well as islet dysfunction in culture. The β-cell specific SCA B1-mediated in vivo delivery of the NBD peptide induced a striking resistance to the development of diabetes in MLDS-injected mice, a murine model for immune-mediated diabetes (Eldor R et al., Proc Natl Acad Sci USA., 103(13):5072-7, 2006). Moreover, the SCA B1-NBD treated mice showed a well-preserved β-cell mass and normal post-challenge plasma concentrations of glucose and insulin following intraperitoneal glucose injection, at 28 days after the last injection of STZ.

Taken together, these data provide strong evidence that the SCA B1-NBD fusion can provide highly specific, targeted therapy to β-cells in vivo, and provides a novel, non-invasive strategy of SCA-mediated β-cell specific delivery, for use in therapeutic interventions to prevent β-cell death in the early stages of diabetes development or islet transplantation. As noted earlier, the SCA-mediated in vivo transport provided herein has clear advantages over strategies reported previously, e.g. adenoviral-mediated transport (Rehman K K et al., J Biol Chem 278(11):9862-8, 2003; Giannoukakis Net al., J. Biol. Chem. 275(47):36509-13, 2000).

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosures as come within known or customary practice within the art to which the disclosure pertains and as may be applied to the essential features herein before set forth, and as follows in the scope of the appended claims.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

The contents of all documents and references cited herein are hereby incorporated by reference in their entirety,

TABLE OF SEQUENCES:
Islet Cell Specific Phage Clone 1-SCA B1
DNA Sequence (SEQ ID NO: 1)
GCCATGGCCGAGGTGCAGCTGTTGGAGTCTGGGGGAGGCTTGGTACAG
CCTGGGGGGTCCCTGAGACTCTCCTGTGCAGCCTCTGGATTCACCTTTA
GCAGCTATGCCATGAGCTGGGTCCGCCAGGCTCCAGGGAAGGGGCTGG
AGTGGGTCTCATCTATTACTGCTGAGGGTACGCATACATGGTACGCAGA
CTCCGTGAAGGGCCGGTTCACCATCTCCAGAGACAATTCCAAGAACACG
CTGTATCTGCAAATGAACAGCCTGAGAGCCGAGGACACGGCCGTATATT
ACTGTGCGAAAACGTCTTATCGGTTTGACTACTGGGGCCAGGGAACCCT
GGTCACCGTCTCGAGCGGTGGAGGCGGTTCAGGCGGAGGTGGCAGCG
GCGGTGGCGGGTCGACGGACATCCAGATGACCCAGTCTCCATCCTCCC
TGTCTGCATCTGTAGGAGACAGAGTCACCATCACTTGCCGGGCAAGTCA
GAGCATTAGCAGCTATTTAAATTGGTATCAGCAGAAACCAGGGAAAGCC
CCTAAGCTCCTGATCTATAAGGCATCCCGTTTGCAAAGTGGGGTCCCAT
CAAGGTTCAGTGGCAGTGGATCTGGGACAGATTTCACTCTCACCATCAG
CAGTCTGCAACCTGAAGATTTTGCAACTTACTACTGTCAACAGAAGTGGG
ATCCTCCTCGGACGTTCGGCCAAGGGACCAAGGTGGAAATCAAACGGG
CGGCCGCACATCATCATCAC
Amino-acid Sequence (SEQ ID NO: 2)
A M A E V Q L L E S G G G L V Q P G G S L R L S C A A S
G F T F S S Y A M S W V R Q A P G K G L E W V S S I T A
E G T H T W Y A D S V K G R F T I S R D N S K N T L Y L
Q M N S L R A E D T A V Y Y C A K T S Y R F D Y W G Q
G T L V T V S S G G G G S G G G G S G G G G S T D I Q
M T Q S P S S L S A S V G D R V T I T C R A S Q S I S S Y
L N W Y Q Q K P G K A P K L L I Y K A S R L Q S G V P S
R F S G S G S G T D F T L T I S S L Q P E D F A T Y Y C
Q Q K W D P P R T F G Q G T K V E I K R A A A H H H H H
H G A A E Q K L I S E E D L N
Islet Cell Specific Phage Clone 2-SCA B2
DNA Sequence (SEQ ID NO: 3)
GCCATGGCCGAGGTGCAGCTGTTGGAGTCTGGGGGAGGCTTGGTACAG
CCTGGGGGGTCCCTGAGACTCTCCTGTGCAGCCTCTGGATTCACCTTTA
GCAGCTATGCCATGAGCTGGGTCCGCCAGGCTCCAGGGAAGGGGCTGG
AGTGGGTCTCACGGATTAAGATTTTTGGTTCGAAGACAAAGTTCGCAGAC
TCCGTGAAGGGCCGGTTCACCATCTCCAGAGACAATTCCAAGAACACGC
TGTATCTGCAAATGAACAGCCTGAGAGCCGAGGACACGGCCGTATATTA
CTGTGCGAAACATTCTACGCATTTTGACTACTGGGGCCAGGGAACCCTG
GTCACCGTCTCGAGCGGTGGAGGCGGTTCAGGCGGAGGTGGCAGCGG
CGGTGGCGGGTCGACGGACATCCAGATGACCCAGTCTCCATCCTCCCT
GTCTGCATCTGTAGGAGACAGAGTCACCATCACTTGCCGGGCAAGTCAG
AGCATTAGCAGCTATTTAAATTGGTATCAGCAGAAACCAGGGAAAGCCC
CTAAGCTCCTGATCTATAGGGCATCCAGTTTGCAAAGTGGGGTCCCATC
AAGGTTCAGTGGCAGTGGATCTGGGACAGATTTCACTCTCACCATCAGC
AGTCTGCAACCTGAAGATTTTGCAACTTACTACTGTCAACAGCTTCAGAG
TACTCCTAGGACGTTCGGCCAAGGGACCAAGGTGGAAATCAAACGGGC
GGCCGCACATCATCATCATCACCATCACGGGGCCGCAGAACAAAAACTC
ATCTCAGGAGAGGATCTGAAT
Amino-acid Sequence (SEQ ID NO: 4)
A M A E V Q L L E S G G G L V Q P G G S L R L S C A A S
G F T F S S Y A M S W V R Q A P G K G L E W V S R I K I
F G S K T K F A D S V K G R F T I S R D N S K N T L Y L
Q M N S L R A E D T A V Y Y C A K H S T H F D Y W G Q
G T L V T V S S G G G G S G G G G S G G G G S T D I Q
M T Q S P S S L S A S V G D R V T I T C R A S Q S I S S Y
L N W Y Q Q K P G K A P K L L I Y R A S S L Q S G V P S
R F S G SG S G T D F T L T I S S L Q P E D F A T Y Y C
Q Q L Q S T P R T F G Q G T K V E I K R A A A H H H H H
H G A A E Q K L I S E E D L N
Islet Cell Specific Phage Clone 5-SCA B3
DNA Sequence (SEQ ID NO: 5)
GCCATGGCCGAGGTGCAGCTGTTGGAGTCTGGGGGAGGCTTGGTACAG
CCTGGGGGGTCCCTGAGACTCTCCTGTGCAGCCTCTGGATTCACCTTTA
GCAGCTATGCCATGAGCTGGGTCCGCCAGGCTCCAGGGAAGGGGCTGG
AGTGGGTCTCATCGATTCATCCTAAGGGTTACCCTACACGGTACGCAGA
CTCCGTGAAGGGCCGGTTCACCATCTCCAGAGACAATTCCAAGAACACG
CTGTATCTGCAAATGAACAGCCTGAGAGCCGAGGACACGGCCGTATATT
ACTGTGCGAAATCGACGACTCCTTTTGACTACTGGGGCCAGGGAACCCT
GGTCACCGTCTCGAGCGGTGGAGGCGGTTCAGGCGGAGGTGGCAGCG
GCGGTGGCGGGTCGACGGACATCCAGATGACCCAGTCTCCATCCTCCC
TGTCTGCATCTGTAGGAGACAGAGTCACCATCACTTGCCGGGCAAGTCA
GAGCATTAGCAGCTATTTAAATTGGTATCAGCAGAAACCAGGGAAAGCC
CCTAAGCTCCTGATCTATGCTGCATCCTCTTTGCAAAGTGGGGTCCCATC
AAGGTTCAGTGGCAGTGGATCTGGGACAGATTTCACTCTCACCATCAGC
AGTCTGCAACCTGAAGATTTTGCAACTTACTACTGTCAACAGATGGGGAG
GGATCCTAGGACGTTCGGCCAAGGGACCAAGGTGGAAATCAAACGGGC
GGCCGCACATCATCATCATCACCATCACGGGGCCGCAGAACAAAAACTC
ATCTCAGGAGAGGATCTGAAT
Amino-acid Sequence (SEQ ID NO: 6)
A M A E V Q L L E S G G G L V Q P G G S L R L S C A A S
G F T F S S Y A M S W V R Q A P G K G L E W V S S I H P
K G Y P T R Y A D S V K G R F T I S R D N S K N T L Y L
Q M N S L R A E D T A V Y Y C A K S T T P F D Y W G Q G
T L V T V S S G G G G S G G G G S G G G G S T D I Q M
T Q S P S S L S A S V G D R V T I T C R A S Q S I S S Y L
N W Y Q Q K P G K A P K L L I Y A A S S L Q S G V P S R
F S G S G S G T D F T L T I S S L Q P E D F A T Y Y C Q
Q M G R D P R T F G Q G T K V E I K R A A A H H H H H H
G A A E Q K L I S E E D L N
Islet Cell Specific Phage Clone 6-SCA B4
DNA Sequence (SEQ ID NO: 7)
GCCATGGCCGAGGTGCAGCTGTTGGAGTCTGGGGGAGGCTTGGTACAG
CCTGGGGGGTCCCTGAGACTCTCCTGTGCAGCCTCTGGATTCACCTTTA
GCAGCTATGCCATGAGCTGGGTCCGCCAGGCTCCAGGGAAGGGGCTGG
AGTGGGTCTCAAGGATTCAGTTTTTTGGTTCGCATACATACTTCGCAGAC
TCCGTGAAGGGCCGGTTCACCATCTCCAGAGACAATTCCAAGAACACGC
TGTATCTGCAAATGAACAGCCTGAGAGCCGAGGACACGGCCGTATATTA
CTGTGCGAAACATTCGACGCATTTTGATTACTGGGGCCAGGGAACCCTG
GTCACCGTCTCGAGCGGTGGAGGCGGTTCAGGCGGAGGTGGCAGCGG
CGGTGGCGGGTCGACGGACATCCAGATGACCCAGTCTCCATCCTCCCT
GTCTGCATCTGTAGGAGACAGAGTCACCATCACTTGCCGGGCAAGTCAG
AGCATTAGCAGCTATTTAAATTGGTATCAGCAGAAACCAGGGAAAGCCC
CTAAGCTCCTGATCTATAGGGCATCCATTTTGCAAAGTGGGGTCCCATCA
AGGTTCAGTGGCAGTGGATCTGGGACAGATTTCACTCTCACCATCAGCA
GTCTGCAACCTGAAGATTTTGCAACTTACTACTGTCAACAGAATAGGAGA
ATTCCTAGGACGTTCGGCCAAGGGACCAAGGTGGAAATCAAACGGGCG
GCCGCACATCATCATCATCACCATCACGGGGCCGCAGAACAAAAACTCA
TCTCAGGAGAGGATCTGAAT
Amino-acid Sequence (SEQ ID NO: 8)
A M A E V Q L L E S G G G L V Q P G G S L R L S C A A S
G F T F S S Y A M S W V R Q A P G K G L E W V S R I Q F
F G S H T Y F A D S V K G R F T I S R D N S K N T L Y L
Q M N S L R A E D T A V Y Y C A K H S T H F D Y W G Q
G T L V T V S S G G G G S G G G G S G G G G S T D I Q
M T Q S P S S L S A S V G D R V T I T C R A S Q S I S S Y
L N W Y Q Q K P G K A P K L L I Y R A S I L Q S G V P S
R F S G S G S G T D F T L T I S S L Q P E D F A T Y Y C Q
Q N R R I P R T F G Q G T K V E I K R A A A H H H H H H
G A A E Q K L I S E E D L N
Islet Cell Specific Phage Clone 7-SCA B5
DNA Sequence (SEQ ID NO: 9)
GCCATGGCCGAGGTGCAGCTGTTGGAGTCTGGGGGAGGCTTGGTACAG
CCTGGGGGGTCCCTGAGACTCTCCTGTGCAGCCTCTGGATTCACCTTTA
GCAGCTATGCCATGAGCTGGGTCCGCCAGGCTCCAGGGAAGGGGCTGG
AGTGGGTCTCATCTATTAGTTCTACTGGTGATTCTACAAGTTACGCAGAC
TCCGTGAAGGGCCGGTTCACCATCTCCAGAGACAATTCCAAGAACACGC
TGTATCTGCAAATGAACAGCCTGAGAGCCGAGGACACGGCCGTATATTA
CTGTGCGAAAGCTGCTGATAGTTTTGACTACTGGGGCCAGGGAACCCTG
GTCACCGTCTCGAGCGGTGGAGGCGGTTCAGGCGGAGGTGGCAGCGG
CGGTGGCGGGTCGACGGACATCCAGATGACCCAGTCTCCATCCTCCCT
GTCTGCATCTGTAGGAGACAGAGTCACCATCACTTGCCGGGCAAGTCAG
AGCATTAGCAGCTATTTAAATTGGTATCAGCAGAAACCAGGGAAAGCCC
CTAAGCTCCTGATCTATGGTGCATCCTCTTTGCAAAGTGGGGTCCCATCA
AGGTTCAGTGGCAGTGGATCTGGGACAGATTTCACTCTCACCATCAGCA
GTCTGCAACCTGAAGATTTTGCAACTTACTACTGTCAACAGACTAATGGT
GCTCCTACTACGTTCGGCCAAGGGACCAAGGTGGAAATCAAACGGGCG
GCCGCACATCATCATCACCATCACGGGGCCGCAGAACAAAAACTCATCT
CAGGAGAGGATCTGAAT
Amino-acid Sequence (SEQ ID NO: 10)
A M A E V Q L L E S G G G L V Q P G G S L R L S C A A S
G F T F S S Y A M S W V R Q A P G K G L E W V S S I S S
T G D S T S Y A D S V K G R F T I S R D N S K N T L Y L
Q M N S L R A E D T A V Y Y C A K A A D S F D Y W G Q
G T L V T V S S G G G G S G G G G S G G G G S T D I Q
M T Q S P S S L S A S V G D R V T I T C R A S Q S I S S Y
L N W Y Q Q K P G K A P K L L I Y G A S S L Q S G V P S
R F S G S G S G T D F T L T I S S L Q P E D F A T Y Y C Q
Q T N G A P T T F G Q G T K V E I K R A A A H H H H H H
G A A E Q K L I S E E D L N
Islet Cell Specific Phage Clone 3-SCA Al
DNA Sequence (SEQ ID NO: 11)
ATGGCCGAGGTGCAGCTGTTGGAGTCTGGGGGAGGCTTGGTACAGCCT
GGGGGGTCCCTGAGACTCTCCTGTGCAGCCTCTGGATTCACCTTTAGCA
GCTATGCCATGAGCTGGGTCCGCCAGGCTCCAGGGAAGGGGCTGGAGT
GGGTCTCACGGATTAGTGTGGCTGGTCGGCGGACAGCTTACGCAGACT
CCGTGAAGGGCCGGTTCACCATCTCCAGAGACAATTCCAAGAACACGCT
GTATCTGCAAATGAACAGCCTGAGAGCCGAGGACACGGCCGTATATTAC
TGTGCGAAAAAGCGGCCTCCGTTTGACTACTGGGGCCAGGGAACCCTG
GTCACCGTCTCGAGCGGTGGAGGCGGTTCAGGCGGAGGTGGCAGCGG
CGGTGGCGGGTCGACGGACATCCAGATGACCCAGTCTCCATCCTCCCT
GTCTGCATCTGTAGGAGACAGAGTCACCATCACTTGCCGGGCAAGTCAG
AGCATTAGCAGCTATTTAAATTGGTATCAGCAGAAACCAGGGAAAGCCC
CTAAGCTCCTGATCTATGCTGCATCCTCTTTGCAAAGTGGGGTCCCATCA
AGGTTCAGTGGCAGTGGATCTGGGACAGATTTCACTCTCACCATCAGCA
GTCTGCAACCTGAAGATTTTGCAACTTACTACTGTCAACAGATGGGGAG
GGATCCTAGGACGTTCGGCCAAGGGACCAAGGTGGAAATCAAACGGGC
GGCCGCACATCATCATCACCAT
Amino-acid Sequence (SEQ ID NO: 12)
A M A E V Q L L E S G G G L V Q P G G S L R L S C A A S
G F T F S S Y A M S W V R Q A P G K G L E W V S R I S V
A G R R T A Y A D S V K G R F T I S R D N S K N T L Y L
Q M N S L R A E D T A V Y Y C A K K R P P F D Y W G Q
G T L V T V S S G G G G S G G G G S G G G G S T D I Q
M T Q S P S S L S A S V G D R V T I T C R A S Q S I S S Y
L N W Y Q Q K P G K A P K L L I Y A A S S L Q S G V P S
R F S G S G S G T D F T L T I S S L Q P E D F A T Y Y C
Q Q M G R D P R T F G Q G T K V E I K R A A A H H H H H
H G A A E Q K L I S E E D L N
Islet Cell Specific Phage Clone 4-SCA A2
DNA Sequence (SEQ ID NO: 13)
GCCATGGCCGAGGTGCAGCTGTTGGAGTCTGGGGGAGGCTTGGTACAG
CCTGGGGGGTCCCTGAGACTCTCCTGTGCAGCCTCTGGATTCACCTTTA
GCAGCTATGCCATGAGCTGGGTCCGCCAGGCtCCAGGGAAGGGGCTGG
AGTGGGTCTCACCTATTGCGTCGCGGGGTGCTCGGACAAATTACGCAGA
CTCCGTGAAGGGCCGGTTCACCATCTCCAGAGACAATTCCAAGAACACG
CTGTATCTGCAAATGAACAGCCTGAGAGCCGAGGACACGGCCGTATATT
ACTGTGCGAAAAAGCCTAGTAGTTTTGACTACTGGGGCCAGGGAACCCT
GGTCACCGTCTCGAGCGGTGGAGGCGGTTCAGGCGGAGGTGGCAGCG
GCGGTGGCGGGTCGACGGACATCCAGATGACCCAGTCTCCATCCTCCC
TGTCTGCATCTGTAGGAGACAGAGTCACCATCACTTGCCGGGCAAGTCA
GAGCATTAGCAGCTATTTAAATTGGTATCAGCAGAAACCAGGGAAAGCC
CCTAAGCTCCTGATCTATAAGGCATCCCCTTTGCAAAGTGGGGTCCCAT
CAAGGTTCAGTGGCAGTGGATCTGGGACAGATTTCACTCTCACCATCAG
CAGTCTGCAACCTGAAGATTTTGCAACTTACTACTGTCAACAGTCGATGC
AGGTTCCTTCTACGTTCGGCCAAGGGACCAAGGTGGAAATCAAACGGGC
GGCCGCACATCATCATCATCACCATCACGGGGCCGCAGAACAAAAACTC
ATCTCAGGAGAGGATCTGAAT
Amino-acid Sequence (SEQ ID NO: 14)
A M A E V Q L L E S G G G L V Q P G G S L R L S C A A S
G F T F S S Y A M S W V R Q A P G K G L E W V S P I A S
R G A R T N Y A D S V K G R F T I S R D N S K N T L Y L
Q M N S L R A E D T A V Y Y C A K K P S S F D Y W G Q
G T L V T V S S G G G G S G G G G S G G G G S T D I Q
M T Q S P S S L S A S V G D R V T I T C R A S Q S I S S Y
L N W Y Q Q K P G K A P K L L I Y K A S P L Q S G V P S
R F S G S G S G T D F T L T I S S L Q P E D F A T Y Y C
Q Q S M Q V P S T F G Q G T K V E I K R A A A H H H H H
H G A A E Q K L I S E E D L N
SCA B1-NBD fusion protein
Amino-acid Sequence (SEQ ID NO: 15)
A M A E V Q L L E S G G G L V Q P G G S L R L S C A A S
G F T F S S Y A M S W V R Q A P G K G L E W V S S I T A
E G T H T W Y A D S V K G R F T I S R D N S K N T L Y L
Q M N S L R A E D T A V Y Y C A K T S Y R F D Y W G Q
G T L V T V S S G G G G S G G G G S G G G G S T D I Q
M T Q S P S S L S A S V G D R V T I T C R A S Q S I S S Y
L N W Y Q Q K P G K A P K L L I Y K A S R L Q S G V P S
R F S G S G S G T D F T L T I S S L Q P E D F A T Y Y C
Q Q K W D P P R T F G Q G T K V E I K R A A A G G G S G
G G T A L D W S W L Q T E H H H H H H
NBD peptide
Amino-acid Sequence (SEQ ID NO: 16)
T A L D W S W L Q T E
Linker peptide
Amino-acid Sequence (SEQ ID NO: 17)
G G G S G G G
SCA B1-NBA fusion protein, without His taq
Amino-acid Sequence (SEQ ID NO: 18)
A M A E V Q L L E S G G G L V Q P G G S L R L S C A A S
G F T F S S Y A M S W V R Q A P G K G L E W V S S I T A
E G T H T W Y A D S V K G R F T I S R D N S K N T L Y L
Q M N S L R A E D T A V Y Y C A K T S Y R F D Y W G Q
G T L V T V S S G G G G S G G G G S G G G G S T D I Q
M T Q S P S S L S A S V G D R V T I T C R A S Q S I S S Y
L N W Y Q Q K P G K A P K L L I Y K A S R L Q S G V P S
R F S G S G S G T D F T L T I S S L Q P E D F A T Y Y C
Q Q K W D P P R T F G Q G T K V E I K R A A A G G G S G
G G T A L D W S W L Q T E

What is claimed is:

1. A fusion protein comprising a single chain antibody (SCA) specifically binding to a cell type of the islets of Langerhans and an agent for the prevention or treatment of a pancreatic condition, or an analog, homolog, fragment or variant thereof which retains the binding specificity of the fusion protein.

2. The fusion protein of claim 1, wherein the agent inhibits NF-κB activation.

3. The fusion protein of claim 2, wherein the agent is a NEMO-Binding Domain (NBD) peptide.

4. The fusion protein of claim 3, wherein the sequence of the NBD peptide comprises the amino acid sequence of SEQ ID NO: 16.

5. The fusion protein of claim 1, wherein the sequence of the SCA comprises:
i) a heavy chain CDR1 amino acid sequence, CDR2 amino acid sequence, and CDR3 amino acid sequence, respectively, of amino acid residues 34-38, 53-69, 102-108 of SEQ ID NO:2; and
ii) a light chain CDR1 amino acid sequence, CDR2 amino acid sequence, and CDR3 amino acid sequence, respectively of amino acid residues 159-169, 185-191, 224-232 of SEQ ID NO:2.

6. The fusion protein of claim 1, wherein the SCA comprises heavy and light chain CDR1, CDR2 and CDR3 amino acid sequences 34-38, 53-69, 102-108, 159-169, 185-191, and 224-232, respectively, of SEQ ID NO:4, SEQ ID NO:6, SEQ ID NO:8, or SEQ ID NO:10, or an analog, homolog, fragment or variant thereof, and specifically binds to a β-cell.

7. The fusion protein of claim 1, wherein the fusion protein specifically binds to a β-cell and selectively inhibits NF-κB activation in the β-cell in vivo.

8. The fusion protein of claim 1, wherein said β cell is identified in a subject, and said subject is a rodent, canine, pig, primate or human.

9. An analog, homolog, fragment or variant of the fusion protein of claim 1, wherein the analog, homolog, fragment or variant has at least 60%, at least 70%, at least 75%, at least 80%, at least 85%, at least 90%, at least 95% or at least 99% sequence identity to the fusion protein.

10. The analog, homolog, fragment or variant of the fusion protein of claim 9, wherein the analog, homolog, fragment or variant retains the binding specificity of the fusion protein.

11. A fusion protein comprising the amino acid sequence set forth in SEQ ID NO: 15 or SEQ ID NO: 18, or an analog, homolog, fragment or variant thereof which retains the binding specificity of the fusion protein.

12. A fusion protein consisting of the amino acid sequence of SEQ ID NO: 15 or SEQ ID NO: 18.

13. The fusion protein of claim 1, further comprising a linker sequence between the SCA and the agent.

14. The fusion protein of claim 13, wherein the linker sequence comprises the amino acid sequence set forth in SEQ ID NO: 17.

15. The fusion protein of claim 1, wherein the SCA is humanized.

16. A nucleic acid molecule comprising a nucleic acid sequence encoding the fusion protein or analog, homolog, fragment or variant thereof of claim 1.

17. The nucleic acid molecule of claim 16 operably linked to an expression control sequence to form an expression vector, wherein said expression vector is propagated in a suitable cell.

18. A pharmaceutical composition comprising the fusion protein or analog, homolog, fragment or variant thereof of claim 1 and a pharmaceutically acceptable carrier or excipient.

19. A method for preventing or treating a pancreatic condition or disease comprising administering the fusion protein or analog, homolog, fragment or variant thereof of claim 1 to a subject in need thereof.

20. The method of claim 19, wherein the condition or disease is a metabolic disorder.

21. The method of claim 19, wherein the condition or disease is a β-cell associated disorder.

22. The method of claim 19, wherein the condition or disease is Type I diabetes, Type II diabetes or a complication of diabetes.

23. The method of claim 19, wherein the condition or disease is an endocrine tumor.

24. The method of claim 19, wherein β-cell degeneration is prevented or inhibited in the subject.

25. The method of claim 19, wherein the functionality of pancreatic cells is improved or restored in the subject.

26. The method of claim 19, wherein plasma insulin levels are increased in the subject.

27. The method of claim 19, wherein the number or size of pancreatic cells is increased in the subject.

28. The method of claim 25 or 27, wherein the pancreatic cells are pancreatic β-cells.

29. The method of claim 19, wherein NF-κB activation is inhibited in the subject.

30. The method of claim 29, wherein NF-κB activation is selectively inhibited in pancreatic β-cells in the subject.

31. The method of claim 30, wherein IL-1β-mediated induction of NF-κB is selectively inhibited in pancreatic β-cells in the subject.

32. The method of claim 19, wherein said polypeptide or analog, homolog, fragment or variant thereof is administered by injection, orally, intravenously, intraperitoneally, intramuscularly or subcutaneously.

33. The method of claim 19, wherein the fusion protein comprises the amino acid sequence set forth in SEQ ID NO: 15 or SEQ ID NO: 18, or an analog, homolog, fragment or variant thereof which retains the binding specificity of the fusion protein.

34. The method of claim 19, wherein the fusion protein consists of the amino acid sequence set forth in SEQ ID NO: 15 or SEQ ID NO: 18.

35. The method of claim 19, wherein the subject is a human.


SEQUENCE LISTING (18 sequences)

SEQ ID NO: 1 - DNA, 750 bp, homo sapiens
gccatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctattactgctgagggtacgcatacatggtacgcagactccgtgaagggccggttcaccatctccagagacaattccaagaacacgctgtatctgcaaatgaacagcctgagagccgaggacacggccgtatattactgtgcgaaaacgtcttatcggtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctataaggcatcccgtttgcaaagtggggtcccatcaaggttcagtggcagtggatctgggacagatttcactctcaccatcagcagtctgcaacctgaagattttgcaacttactactgtcaacagaagtgggatcctcctcggacgttcggccaagggaccaaggtggaaatcaaacgggcggccgcacatcatcatcac

SEQ ID NO: 2 - protein, 266 aa, homo sapiens
AMAEVQLLESGGGLVQPGGSLRLSCAASGFTFSSYAMSWVRQAPGKGLEWVSSITAEGTHTWYADSVKGRFTISRDNSKNTLYLQMNSLRAEDTAVYYCAKTSYRFDYWGQGTLVTVSSGGGGSGGGGSGGGGSTDIQMTQSPSSLSASVGDRVTITCRASQSISSYLNWYQQKPGKAPKLLIYKASRLQSGVPSRFSGSGSGTDFTLTISSLQPEDFATYYCQQKWDPPRTFGQGTKVEIKRAAAHHHHHHGAAEQKLISEEDLN

SEQ ID NO: 3 - DNA, 801 bp, homo sapiens
gccatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcacggattaagatttttggttcgaagacaaagttcgcagactccgtgaagggccggttcaccatctccagagacaattccaagaacacgctgtatctgcaaatgaacagcctgagagccgaggacacggccgtatattactgtgcgaaacattctacgcattttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatccagtttgcaaagtggggtcccatcaaggttcagtggcagtggatctgggacagatttcactctcaccatcagcagtctgcaacctgaagattttgcaacttactactgtcaacagcttcagagtactcctaggacgttcggccaagggaccaaggtggaaatcaaacgggcggccgcacatcatcatcatcaccatcacggggccgcagaacaaaaactcatctcaggagaggatctgaat

SEQ ID NO: 4 - protein, 266 aa, homo sapiens
AMAEVQLLESGGGLVQPGGSLRLSCAASGFTFSSYAMSWVRQAPGKGLEWVSRIKIFGSKTKFADSVKGRFTISRDNSKNTLYLQMNSLRAEDTAVYYCAKHSTHFDYWGQGTLVTVSSGGGGSGGGGSGGGGSTDIQMTQSPSSLSASVGDRVTITCRASQSISSYLNWYQQKPGKAPKLLIYRASSLQSGVPSRFSGSGSGTDFTLTISSLQPEDFATYYCQQLQSTPRTFGQGTKVEIKRAAAHHHHHHGAAEQKLISEEDLN

SEQ ID NO: 5 - DNA, 801 bp, homo sapiens
gccatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcatcgattcatcctaagggttaccctacacggtacgcagactccgtgaagggccggttcaccatctccagagacaattccaagaacacgctgtatctgcaaatgaacagcctgagagccgaggacacggccgtatattactgtgcgaaatcgacgactccttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatcctctttgcaaagtggggtcccatcaaggttcagtggcagtggatctgggacagatttcactctcaccatcagcagtctgcaacctgaagattttgcaacttactactgtcaacagatggggagggatcctaggacgttcggccaagggaccaaggtggaaatcaaacgggcggccgcacatcatcatcatcaccatcacggggccgcagaacaaaaactcatctcaggagaggatctgaat

SEQ ID NO: 6 - protein, 266 aa, homo sapiens
AMAEVQLLESGGGLVQPGGSLRLSCAASGFTFSSYAMSWVRQAPGKGLEWVSSIHPKGYPTRYADSVKGRFTISRDNSKNTLYLQMNSLRAEDTAVYYCAKSTTPFDYWGQGTLVTVSSGGGGSGGGGSGGGGSTDIQMTQSPSSLSASVGDRVTITCRASQSISSYLNWYQQKPGKAPKLLIYAASSLQSGVPSRFSGSGSGTDFTLTISSLQPEDFATYYCQQMGRDPRTFGQGTKVEIKRAAAHHHHHHGAAEQKLISEEDLN

SEQ ID NO: 7 - DNA, 801 bp, homo sapiens
gccatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcaaggattcagttttttggttcgcatacatacttcgcagactccgtgaagggccggttcaccatctccagagacaattccaagaacacgctgtatctgcaaatgaacagcctgagagccgaggacacggccgtatattactgtgcgaaacattcgacgcattttgattactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatccattttgcaaagtggggtcccatcaaggttcagtggcagtggatctgggacagatttcactctcaccatcagcagtctgcaacctgaagattttgcaacttactactgtcaacagaataggagaattcctaggacgttcggccaagggaccaaggtggaaatcaaacgggcggccgcacatcatcatcatcaccatcacggggccgcagaacaaaaactcatctcaggagaggatctgaat

SEQ ID NO: 8 - protein, 266 aa, homo sapiens
AMAEVQLLESGGGLVQPGGSLRLSCAASGFTFSSYAMSWVRQAPGKGLEWVSRIQFFGSHTYFADSVKGRFTISRDNSKNTLYLQMNSLRAEDTAVYYCAKHSTHFDYWGQGTLVTVSSGGGGSGGGGSGGGGSTDIQMTQSPSSLSASVGDRVTITCRASQSISSYLNWYQQKPGKAPKLLIYRASILQSGVPSRFSGSGSGTDFTLTISSLQPEDFATYYCQQNRRIPRTFGQGTKVEIKRAAAHHHHHHGAAEQKLISEEDLN

SEQ ID NO: 9 - DNA, 798 bp, homo sapiens
gccatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctattagttctactggtgattctacaagttacgcagactccgtgaagggccggttcaccatctccagagacaattccaagaacacgctgtatctgcaaatgaacagcctgagagccgaggacacggccgtatattactgtgcgaaagctgctgatagttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatcctctttgcaaagtggggtcccatcaaggttcagtggcagtggatctgggacagatttcactctcaccatcagcagtctgcaacctgaagattttgcaacttactactgtcaacagactaatggtgctcctactacgttcggccaagggaccaaggtggaaatcaaacgggcggccgcacatcatcatcaccatcacggggccgcagaacaaaaactcatctcaggagaggatctgaat

SEQ ID NO: 10 - protein, 266 aa, homo sapiens
AMAEVQLLESGGGLVQPGGSLRLSCAASGFTFSSYAMSWVRQAPGKGLEWVSSISSTGDSTSYADSVKGRFTISRDNSKNTLYLQMNSLRAEDTAVYYCAKAADSFDYWGQGTLVTVSSGGGGSGGGGSGGGGSTDIQMTQSPSSLSASVGDRVTITCRASQSISSYLNWYQQKPGKAPKLLIYGASSLQSGVPSRFSGSGSGTDFTLTISSLQPEDFATYYCQQTNGAPTTFGQGTKVEIKRAAAHHHHHHGAAEQKLISEEDLN

SEQ ID NO: 11 - DNA, 750 bp, homo sapiens
atggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcacggattagtgtggctggtcggcggacagcttacgcagactccgtgaagggccggttcaccatctccagagacaattccaagaacacgctgtatctgcaaatgaacagcctgagagccgaggacacggccgtatattactgtgcgaaaaagcggcctccgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatcctctttgcaaagtggggtcccatcaaggttcagtggcagtggatctgggacagatttcactctcaccatcagcagtctgcaacctgaagattttgcaacttactactgtcaacagatggggagggatcctaggacgttcggccaagggaccaaggtggaaatcaaacgggcggccgcacatcatcatcaccat

SEQ ID NO: 12 - protein, 266 aa, homo sapiens
AMAEVQLLESGGGLVQPGGSLRLSCAASGFTFSSYAMSWVRQAPGKGLEWVSRISVAGRRTAYADSVKGRFTISRDNSKNTLYLQMNSLRAEDTAVYYCAKKRPPFDYWGQGTLVTVSSGGGGSGGGGSGGGGSTDIQMTQSPSSLSASVGDRVTITCRASQSISSYLNWYQQKPGKAPKLLIYAASSLQSGVPSRFSGSGSGTDFTLTISSLQPEDFATYYCQQMGRDPRTFGQGTKVEIKRAAAHHHHHHGAAEQKLISEEDLN

SEQ ID NO: 13 - DNA, 801 bp, homo sapiens
gccatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcacctattgcgtcgcggggtgctcggacaaattacgcagactccgtgaagggccggttcaccatctccagagacaattccaagaacacgctgtatctgcaaatgaacagcctgagagccgaggacacggccgtatattactgtgcgaaaaagcctagtagttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctataaggcatcccctttgcaaagtggggtcccatcaaggttcagtggcagtggatctgggacagatttcactctcaccatcagcagtctgcaacctgaagattttgcaacttactactgtcaacagtcgatgcaggttccttctacgttcggccaagggaccaaggtggaaatcaaacgggcggccgcacatcatcatcatcaccatcacggggccgcagaacaaaaactcatctcaggagaggatctgaat

SEQ ID NO: 14 - protein, 266 aa, homo sapiens
AMAEVQLLESGGGLVQPGGSLRLSCAASGFTFSSYAMSWVRQAPGKGLEWVSPIASRGARTNYADSVKGRFTISRDNSKNTLYLQMNSLRAEDTAVYYCAKKPSSFDYWGQGTLVTVSSGGGGSGGGGSGGGGSTDIQMTQSPSSLSASVGDRVTITCRASQSISSYLNWYQQKPGKAPKLLIYKASPLQSGVPSRFSGSGSGTDFTLTISSLQPEDFATYYCQQSMQVPSTFGQGTKVEIKRAAAHHHHHHGAAEQKLISEEDLN

SEQ ID NO: 15 - protein, 270 aa, artificial sequence
  synthesized
AMAEVQLLESGGGLVQPGGSLRLSCAASGFTFSSYAMSWVRQAPGKGLEWVSSITAEGTHTWYADSVKGRFTISRDNSKNTLYLQMNSLRAEDTAVYYCAKTSYRFDYWGQGTLVTVSSGGGGSGGGGSGGGGSTDIQMTQSPSSLSASVGDRVTITCRASQSISSYLNWYQQKPGKAPKLLIYKASRLQSGVPSRFSGSGSGTDFTLTISSLQPEDFATYYCQQKWDPPRTFGQGTKVEIKRAAAGGGSGGGTALDWSWLQTEHHHHHH

SEQ ID NO: 16 - protein, 11 aa, homo sapiens
TALDWSWLQTE

SEQ ID NO: 17 - protein, 7 aa, artificial sequence
  synthesized
GGGSGGG

SEQ ID NO: 18 - protein, 264 aa, artificial sequence
  synthesized
AMAEVQLLESGGGLVQPGGSLRLSCAASGFTFSSYAMSWVRQAPGKGLEWVSSITAEGTHTWYADSVKGRFTISRDNSKNTLYLQMNSLRAEDTAVYYCAKTSYRFDYWGQGTLVTVSSGGGGSGGGGSGGGGSTDIQMTQSPSSLSASVGDRVTITCRASQSISSYLNWYQQKPGKAPKLLIYKASRLQSGVPSRFSGSGSGTDFTLTISSLQPEDFATYYCQQKWDPPRTFGQGTKVEIKRAAAGGGSGGGTALDWSWLQTE